(19) United States
(12) Patent Application Publication
(10) Pub. No.: US 2015/0369820 A1
(43) Pub. Date: Dec. 24, 2015

(54) NOVEL DISEASE-MARKER

(71) Applicants: AARHUS UNIVERSITET, Aarhus C (DK); REGION MIDTJYLLAND, Viborg (DK)

(72) Inventors: Holger Jon Møller, Risskov (DK); Sidsel Rødgaard-Hansen, Gjern (DK)

(73) Assignees: Aarhus Universitet, Aarhus C (DK); Region Midtjylland, Viborg (DK)

(21) Appl. No.: 14/763,192

(22) PCT Filed: Jan. 24, 2014
(86) PCT No.: PCT/DK2014/050014

§ 371 Date: Jul. 24, 2015

(30) Foreign Application Priority Data
Jan. 25, 2013 (DK) PA 2013 70043
Jan. 25, 2013 (DK) PA 2013 70044

(51) Int. Cl. G01N 33/68; G01N 33/49; G01N 21/75; G01N 30/00; H01J 49/00

(52) U.S. Cl. CPC G01N 33/6872; G01N 30/00; H01J 49/0027; G01N 21/75; G01N 33/49; G01N 2333/70596; G01N 2800/52

(57) ABSTRACT

The invention relates to the use of soluble CD206 as a general health/disease marker, and a specific marker for liver disorders, sepsis, and pneumonia.

All patent and non-patent references cited in the application are hereby incorporated by reference in their entirety.

### FIELD OF INVENTION

The present invention relates to the use of soluble CD206 as a general health/disease marker, and a specific marker for liver disorders, sepsis, and pneumonia.

### BACKGROUND OF INVENTION

The mannose receptor is a C-type lectin carbohydrate binding protein primarily present on the surface of macrophages and dendritic cells. It helps recognize pathogens that have mannose on their surface, and triggers one pathway of the complement system.

The function of this receptor is to recognize complex carbohydrates that are located on glycoproteins that are a part of many different biological processes. Some of those processes include cell—cell recognition, serum glycoprotein turnover, and neutralization of pathogens. The protein also functions as a type 1 membrane immune receptor that mediates the endocytosis of glycoproteins by macrophages. The structure of these proteins allows them to bind to high mannose structures on the surface of potentially pathogenic viruses, bacteria, and fungi so that they can be engulfed by the cell.

Humans have two different mannose receptors, MRC1 and MRC2. MRC1 is also known as CD206.

Arce-Mendoza et al, “Expression of CD64, CD206 and RAGE in adherent cells of diabetic patients infected with Mycobacterium tuberculosis” Arch Med Res 2008; 39(3): 306-11 discloses a study of the expression of CD64, CD206 and RAGE on mononuclear cells isolated and cultured to obtain adherent cells using flow cytometry. Four groups of patients were examined; a group with type 2 diabetes, a group with pulmonary tuberculosis, a group with type 2 diabetes and pulmonary tuberculosis and a control group. The group with type 2 diabetes and the group with type 2 diabetes and pulmonary tuberculosis expressed greater mean fluorescense intensity of CD206 than the control group. The measurements are done on mononuclear cells isolated and cultured to obtain adherent cells and then stimulated with M. tuberculosis H37Rv lipids.

Porcheray et al., “Macrophage activation switching: an asset for the resolution of inflammation” Clin Exp Immunol, 2005; 142(3): 481-9 discloses that macrophage activation leads to differential phenotypes. It is disclosed that CD163 and CD206 exhibits mutually exclusive induction patterns after stimulation by a panel of anti-inflammatory molecules.

Linehan, S A, “The mannose receptor is expressed by subsets of APC in non-lymphoid organs” BMC Immunol, 2005; 6:4 discloses a study identifying the mannose receptor (MR) positive APC's and their distribution in different organs. It is concluded that MR positive APC are present in several peripheral organs like skin, liver, cardiac and skeletal muscle and tongue.

Nguyen and Hildreth, “Involvement of macrophage mannose receptor in the binding and transmission of HIV by macrophages” Eur J Immunol, 2003; 33(2): 483-93 discloses the ability of macrophages to bind HIV and facilitate its transmission to T cells. The initial association of HIV with macrophages is macrophage mannose receptor (MMR) mediated.

Allavena et al. “Engagement of the mannose receptor by tumoral mucins activates an immune suppressive phenotype in human tumor-associated macrophages” Clin Dev Immunol, 2010; Epub 2011 Feb. 9 discloses that Tumor-associated macrophages (TAMs) isolated from human ovarian carcinoma most abundantly expresses the mannose Receptor (MR). It is contemplating that this association may contribute to the immune suppressive phenotype of the TAMs.

Wollenberg et al “Expression and function of the mannose receptor CD206 on epidermal dendritic cells in inflammatory skin diseases” J Invest Dermatol, 2002; 118(2): 327-34 discloses that some epidermal dendritic cells may express CD206 under inflammatory skin conditions. It is contemplated that the inflammatory dendritic epidermal cells use CD206 for receptor-mediated endocytosis.

Martinez-Pomares et al “A functional soluble form of the murine mannose receptor is produced by macrophages in vitro and is present in mouse serum”, J Biol Chem 1998, 273:23376-80 discloses that the extracellular domain of the murine mannose receptor is shed from macrophages and can be detected in mouse serum. According to the reference sMR levels in mouse serum were not affected by Mycobacterium bovis bacillus, Calmette-Guérin or C. albicans infection or by intraperitoneal treatment with LPS, zymosan or heat-killed C. albicans.

The prior art provides no demonstration that soluble CD206 can be detected in human serum. Further, the prior art provides no indication that soluble CD206 can be used as a diagnostic marker. In contrast, according to Martinez-Pomares et al 1998, serum levels of murine CD206 is not affected by bacterial and fungal infections.

### SUMMARY OF INVENTION

The present invention relates to the novel use of the CD206 protein as a sensitive biomarker for predicting the risk of a subject having a disorder and/or disease and is based on the surprising finding that human serum contains substantial amounts of soluble CD206 and that the level of soluble CD206 surprisingly correlates with the subject's health condition. Specifically, the inventors have observed that the amount of soluble CD206 in serum from healthy subjects is relatively low and that the level is significantly increased in subjects in intensive care units, especially in severely ill patients with sepsis, in patients with liver disease and in pneumonia patients.

The level of sCD206 can be used for several purposes. In apparently healthy subjects an increased level of sCD206 is indicative of the presence of a latent or developing disease. Early identification of subjects with latent or early stage disease can be used to improve diagnosis or treatment. If a subject is feeling unwell, an increased level of sCD206 is indicative of the presence of a disease that could be severe and may require further investigation and subsequent treatment.

As the level of sCD206 also correlates with disease severity and with fatal outcome, the marker can be used to predict disease severity in subjects that are sick and optionally have been diagnosed with a disorder. In subjects diagnosed with a disease the level of sCD206 can be used to diagnose or prognose disease progression and to follow the efficacy of any treatment.

In sick subjects, in particular in severely sick subjects, the level of sCD206 can be used to predict fatal outcome of the disease.

The level of sCD206 can therefore be used to distinguish between subjects that require further diagnostic assessment and/or intensified treatment (in particular subjects suspected of having sepsis, a hepatological disorder, or a pneumococcal disorder) and those that do not. As demonstrated in Example 4, the level of sCD206 is increased in invasive pneumococcal disease (IPD) and the level is indicative of fatal outcome.

The inventors have demonstrated that serum sCD206 is significantly increased in subjects with acute alcoholic hepatitis. The level of soluble CD206 in human serum has also been found to correlate at least to some extent with the serum level of CD163. Consequently, the serum level of CD206 may be used as a biomarker to the same extent as soluble CD163.

One preferred embodiment relates to the use of CD206 to predict the risk of having a liver disorder, preferably hepatitis, more preferably hepatitis C cirrhosis or fibrosis, or acute alcoholic hepatitis. In other embodiments, the liver disorder may be fatty liver disease, more specifically hepatitic steatosis.

Another preferred embodiment relates to the use of CD206 to identify patients with sepsis or septic shock among severely ill patients, and to predict the risk of contracting complications, including mortality, in patients with sepsis. In other embodiments, the severe disorder may be SIRS (systemic inflammatory Response Syndrome).

In a further aspect the invention relates to a method of treatment of a disease or disorder comprising treating subjects having an increased level of sCD206. Continued measurements of sCD206 can be used to follow the efficacy of the treatment.

In yet another preferred aspect, the present invention relates to the use of sCD206 in a method of assessing the efficacy of a therapy given to a patient by assessing the level of sCD206 in serum from a patient, subjecting said patient to therapy and subsequently assessing the level of sCD206 again to evaluate whether said therapy is efficious.

In yet another aspect, the present invention relates to the use in a method of assessing the safety of a potential therapy and/or drug candidate by determining the level of sCD206 in a subject, subjecting said subject to said therapy and/or drug candidate and determining the level of sCD206, wherein the absence of a significant increase in sCD206 indicates that the therapy and/or drug candidate is safe.

In yet another aspect, the present invention relates to the use of sCD206 in a method of screening donor blood, wherein the donor blood is evaluated for the content of sCD206 and said contents is compared to a cut-off value indicating whether the subject donating the blood is healthy or not. Thus the method may be used to determine whether the blood is safe enough to be used for transfusion or for preparation of blood-derived products.

In further aspects, the application relates to the use of the CD206 protein as a sensitive biomarker for predicting whether a subject is suffering from a fungal infection and is based on the surprising finding that human serum contains substantial amounts of soluble CD206. The level of sCD206 can therefore be used to distinguish between subjects that suffer from a fungal infection and those that do not.

In a further aspect the invention relates to a method of treatment of a fungal infection comprising treating subjects having an increased level of sCD206. Continued measurements of sCD206 can be used to follow the efficacy of the treatment.

In a further aspect the invention relates to a method of diagnosing and treatment of complications to a fungal infection comprising treating subjects having an increased level of sCD206. Continued measurements of sCD206 can be used to follow the efficacy of the treatment.

In yet another preferred aspect, the present invention relates to the use of sCD206 in a method of assessing the efficacy of a therapy given to a patient suffering from a fungal infection by assessing the level of sCD206 in serum from a patient, subjecting said patient to therapy and subsequently assessing the level of sCD206 again to evaluate whether said therapy is efficacious.

In an aspect, the application relates to a method for diagnosing or prognosing a disease, or the risk thereof, in a subject in need thereof, comprising: (a) determining an amount of soluble CD206 in a biological sample including a biological fluid or a biological tissue from the subject; and (b) comparing the amount of the soluble CD206 in the sample with a control level, wherein if the amount determined in (a) is higher the control level, the subject is diagnosed as having, or at an increased risk of developing, a disease. The disease may be any of the diseases as herein described. The control level may be a level representative of healthy subjects, optionally age or sex corrected or a cutoff level as herein described.

In other aspects the application relates to assessing the severity of a disease, or the risk of fatal outcome, in a subject in need thereof, comprising: (a) determining an amount of soluble CD206 in a biological sample including a biological fluid or a biological tissue from the subject; and (b) comparing the amount of the soluble CD206 in the sample with a control level, wherein if the amount determined in (a) is higher the control level, the severity of the disease is predicted to be higher or even fatal. In these cases the control level may be a level representative of healthy subjects, a level representative of sick subjects with non-severe disease, a cutoff level as herein described, or a level of sCD206 determined in the same subject prior to disease or in an earlier (less severe) stage of the disease.

### FIGURE LEGENDS

FIG. 1. Purification of sMR from human plasma

(A) Immunoblotting of different human plasma samples (lanes 1-4), human macrophage lysate (positive control, lane 5), and bladder cancer cell lysate (negative control, lane 6) with monoclonal mouse anti human sMR. Enhanced chemiluminescence was used as detection system. The concentration of sMR as measured by the sandwich enzyme-linked immunosorbent assay (ELISA) is indicated for each sample.

(B) Coomassie Brilliant Blue staining of purified sMR from human plasma separated by SDS-PAGE. Lanes 1-4 were purified using mannose affinity chromatography, lane 5 by immune-affinity chromatography. Bands corresponding to approximately 170 kDa were cut and analysed by MALDI MS/MS.

FIG. 2. Identification of sMR in human plasma by peptide mapping Schematic representation of the full length mannose receptor. Peptides homologous to human MR identified by MALDI MS/MS in the five bands are indicated by asterixes.

FIG. 3. ELISA for measuring sMR in human plasma

A) Linearity. Serum (twofold diluted 1:20-1:10,240) was measured in quadruplicates.

(B) Comparability of serum vs. plasma. Paired serum, EDTA-plasma, and heparin-plasma was collected from 30 patients and sMR determined in duplicates.

(C) Stability over time. Three pools of fresh plasma were prepared, and aliquots of 50 μl were taken from each pool and kept at RT, +6° C., −20° C. and −80° C. Samples were analyzed during a period of 270 days.

(D) Freeze-thaw stability. Seven aliquots were taken from each of three pools of plasma and frozen (−80° C.) and thawed (+20° C.) one to seven times, frozen at −80° C. and subsequently analyzed in the same run.

FIG. 4. Concentration of sMR in healthy individuals

The concentration of sMR in serum from 217 healthy individuals was measured by ELISA. Reference intervals for intervals<50 years (0.09-0.37 mg/l) and 50 years (0.12-0.46 mg/l) are indicated.

FIG. 5. Correlation of sMR with CRP and sCD163

Comparison of sMR concentrations in 218 hospitalized patients with CRP (A) and sCD163 (B). Dotted lines indicate upper and lower limits of the reference intervals. sMR and sCD163 were measured by ELISA.

FIG. 6. Concentration of sMR in hospitalized patients

Serum concentrations of sMR in healthy compared to hospitalized patients as measured by ELISA. The mean concentrations are indicated. Med: Mainly endocrinological and haematological patients (n=153). The patients in this group have been hospitalized for different durations. Consequently this group covers a rather diverse section of patients, some of which have been cured or have been almost cured.

Hepatol: Hepatological patients (n=37). ICU: patient from intensive care unit (n=28). AAH: Patients with acute alcoholic hepatitis (n=50).

FIG. 7 MR sequences

Sequence alignment of amino acid sequences of CD206 from humans (SEQ ID NO:1), pig (SEQ ID NO:4), mouse (SEQ ID NO:2), rat (SEQ ID NO:3), and Rhesus macaque (SEQ ID NO:5). The alignment prepared in Clustal W illustrates the degree of conservation and provides a consensus sequence for the group of sequences.

FIG. 8: Expression of monocyte-bound CD206 and levels of sCD206 over time in patients with severe sepsis or septic shock, severely ill non-septic patients and healthy controls

Panel A+B: The x-axis represents time (day 1, 2, 3, and 4 of ICU admission), and the y-axis represents the median fluorescence intensity or concentration of sCD163 (mg/L). Dots represent median, bars represent interquartil range. During the four-day observation period, monocyte expression of CD206 (panel A) and soluble CD206 (panel B) was significantly higher in patients with severe sepsis or septic shock compared with severely ill non-septic patients (p<0.001 for both comparisons). At ICU admission, monocyte expression of CD206 and levels of sCD206 were significantly higher in patients with severe sepsis and septic shock compared to severely ill non-septic patients and healthy controls (p<0.001 for all comparison). Levels of sCD206 were higher in the non-septic patients compared to controls (p=0.002). With regards to monocyte expression of CD206, we observed no difference between severely ill non-septic patients and controls.

Panel C: Monocyte expression of CD206 did not correlate with sCD206.

FIG. 9: Area under receiver operating characteristic curve for discrimination between severe sepsis or septic shock and severely ill non-septic patients at ICU admission

At ICU admission sCD206 had the highest AUROC (1; 95% CI 1 to 1), followed by sCD163 (0.95; 95% CI 0.788 to 1), CD206 (0.86; 95% CI 0.73 to 0.98), and CD163 (0.73; 95% CI 0.56 to 0.90).

FIG. 10: Expression of monocyte-bound CD163 and CD206 and levels of sCD163 and sCD206 and area under receiver operating characteristic curve for discrimination between in-hospital survivors and non-survivors Panel A: Monocyte expression of CD163 (p<0.001) and panel B: levels of sCD163 (p=0.004) was significantly higher in the septic patients compared to the non-septic patients.

Panel C: Monocyte expression of CD206 (p=0.02) and panel D: levels of sCD206 (p<0.001) was significantly higher in the septic patients compared to the non-septic patients.

During the 4-day observation period monocyte expression of CD163 (p<0.001) and levels of sCD163 (p=0.004) were significantly higher in the septic patients compared to the non-septic patients (Panel A+B). Monocyte expression of CD206 (p=0.02) and levels of sCD206 (p<0.001) was also observed to besignificantly higher in the septic patients compared to the non-septic patients (Panel C+D).

At ICU admission, monocyte expression of CD163 (p=0.006) and sCD163 (p=0.02) was higher in in-hospital non-survivors than survivors. Likewise, monocyte expression of CD206 (p=0.003) and sCD206 (p=0.001) was also higher in in-hospital non-survivors than survivors.

At ICU admission sCD206 had the highest AUROC (0.87; 95% CI 0.74 to 0.99), followed by CD206 (0.79; 95% CI 0.64 to 0.95), sCD163 (0.78; 95% CI 0.60 to 0.95), and CD163 (0.77; 95% CI 0.60 to 0.95). There were no differences between variables (Panel E).

FIG. 11: Levels of soluble mannose receptor (sMR) in patients with pneumococcal bacteremia in relation to survival.

A. all patients, B Patients below 75 yrs, C. Patients above 75 yrs.

FIG. 12: Receiver operating characteristic curves for the prediction by sMR of fatal outcome in patients with pneumococcal bacteremia

A. all patients, B Patients below 75 yrs, C. Patients above 75 yrs.

FIG. 13: The plasma concentrations of soluble CD163 (sCD163, a) and soluble mannose receptor (sMR, b) were significantly higher in patients with cirrhosis compared to patients with no/mild fibrosis. c Plasma concentrations of sCD163 and sMR correlated significantly for both patients with no/mild fibrosis (open circles) and patients with cirrhosis (solid circles). d In the receiver operating characteristic curve (ROC) analyses for the diagnosis of cirrhosis, the area under the curve (AUC) for sCD163 was higher than for sMR; however, the difference was not significant (Table 8)

### DEFINITIONS

The term CD206 used herein refers to both soluble and membrane-bound forms. CD206 is also known as Mannose receptor, MRC1, Macrophage mannose receptor 1, MMR, C-type lectin domain family 13 member D, C-type lectin domain family 13 member D-like, Macrophage mannose receptor 1-like protein, 1 CD_antigen=CD206,

The term “sCD206”=soluble CD206=shed CD206=plasma CD206=serum CD206=circulating CD206=sMR

The term ‘soluble’ used herein refers to the property of a solid, liquid, or gaseous chemical substance to dissolve in a liquid solvent to form a homogeneous solution. Further it refers to a compound, such as a protein, being in liquid solution as not being attached to a membrane or other anchoring or attaching moieties.

The term ‘disorder’ used herein refers to a medical problem, and is an abnormal condition of an organism that impairs bodily functions, associated with specific symptoms and signs. It may be caused by external factors, such as invading organisms, or it may be caused by internal dysfunctions.

The term ‘disease’ as used herein refers to an abnormal condition of the body or mind that causes discomfort or dysfunction; it is distinct from injury insofar as the latter is usually instantaneously acquired.

The term ‘protein’ used herein refers to an organic compound, also known as a polypeptide, which is a peptide having at least, and preferably more than two amino acids. The generic term amino acid comprises both natural and non-natural amino acids any of which may be in the ‘D’ or ‘L’ isomeric form.

The term ‘biological sample’ used herein refers to any sample selected from the group, but not limited to, serum, plasma, whole blood, saliva, urine, lymph, a biopsy, semen, faeces, tears, sweat, milk, cerebrospinal fluid, ascites fluid, synovial fluid.

The term ‘binding assay’ used herein refers to any biological or chemical assay in which any two or more molecules bind, covalently or non-covalently, to each other thereby enabling measuring the concentration of one of the molecules.

The term ‘chromatographic method’ used herein refers to a collective term for the process of separating mixtures. It involves passing a mixture dissolved in a “mobile phase” through a stationary phase, which separates the analyte to be measured from other molecules in the mixture and allows it to be isolated.

The term ‘risk factor’ used herein refers to a variable associated with an increased risk of disease or infection. Risk factors are correlational and not necessarily causal, because correlation does not imply causation.

The term ‘detection moiety’ used herein refers to a specific part of a molecule, preferably but not limited to be a protein, able to bind and detect another molecule.

The term “subject” used herein refers to a mammal, such as a dog, a cat, a horse, cattle, a camel or a human. In a preferred embodiment the subject is a human.

The term “Sequence identity” used herein refers to the determination of percent identity between two amino acid sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTP program of Altschul, et al. (1990) J. Mol. Biol. 215:403-410.

Another preferred, non-limiting example of a mathematical algorithm utilized for the comparison of sequences is the CLUSTAL W (1.7) alignment algorithm (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680). CLUSTAL W can be used for multiple sequence alignment preferably using BLOSUM 62 as scoring matrix. When calculating sequence identities, CLUSTAL W includes any gaps made by the alignment in the length of the reference sequence. Sequence identities are calculated by dividing the number of matches by the length of the aligned sequences with gaps.

Hepatology is the branch of medicine that incorporates the study of liver, gallbladder, biliary tree, and pancreas as well as management of their disorders. Diseases and complications related to viral hepatitis and alcohol are the main reason for seeking specialist advice. More than 2 billion people have been infected with Hepatitis B virus at some point in their life, and approximately 350 million have become persistent carriers. Up to 80% of liver cancers can be attributed to either Hepatitis B or Hepatitis C virus. In terms of mortality, the former is second only to smoking among known agents causing cancer. With more widespread implementation of vaccination and strict screening before blood transfusion, lower infection rates are expected in the future. In many countries, though, overall alcohol consumption is increasing, and consequently the number of people with cirrhosis and other related complications is commensurately increasing.

According to the present invention liver diseases include the following ICD10 classified liver diseases:

K70 Alcoholic liver disease

Alcoholic fatty liver

Alcoholic hepatitis

Alcoholic fibrosis and sclerosis of liver

Alcoholic cirrhosis of liver
Alcoholic cirrhosis not otherwise specified (NOS)

Alcoholic hepatic failure
NOS
acute
chronic
subacute
with or without hepatic coma

Alcoholic liver disease, unspecified

K71 Toxic liver disease

Toxic liver disease with cholestasis
Cholestasis with hepatocyte injury
“Pure” cholestasis

Toxic liver disease with hepatic necrosis
Hepatic failure (acute), (chronic) due to drugs

Toxic liver disease with acute hepatitis

Toxic liver disease with chronic persistent hepatitis

Toxic liver disease with chronic lobular hepatitis

Toxic liver disease with chronic active hepatitis
Toxic liver disease with lupoid hepatitis

Toxic liver disease with hepatitis, not elsewhere classified

Toxic liver disease with fibrosis and cirrhosis of liver

Toxic liver disease with other disorders of liver
Toxic liver disease with:
focal nodular hyperplasia
hepatic granulomas
peliosis hepatis
veno-occlusive disease of liver

Toxic liver disease, unspecified

K72 Hepatic failure, not elsewhere classified

hepatic:
coma NOS
encephalopathy NOS

hepatitis:
acute
fulminant
malignant

liver (cell) necrosis with hepatic failure

yellow liver atrophy or dystrophy

Acute and subacute hepatic failure

Chronic hepatic failure

Hepatic failure, unspecified

K73 Chronic hepatitis, not elsewhere classified

Chronic persistent hepatitis, not elsewhere classified

Chronic lobular hepatitis, not elsewhere classified

Chronic active hepatitis, not elsewhere classified
Lupoid hepatitis NEC

Other chronic hepatitis, not elsewhere classified

Chronic hepatitis, unspecified

K74 Fibrosis and cirrhosis of liver

Hepatic fibrosis

Hepatic sclerosis

Hepatic fibrosis with hepatic sclerosis

Primary biliary cirrhosis
Chronic nonsuppurative destructive cholangitis

Secondary biliary cirrhosis

Biliary cirrhosis, unspecified

Other and unspecified cirrhosis of liver
Cirrhosis (of liver):
NOS
cryptogenic
macronodular
micronodular
mixed type
portal
postnecrotic

K76 Other diseases of liver

Fatty (change of) liver, not elsewhere classified

Chronic passive congestion of liver
Cardiac:
cirrhosis (so-called) of liver
sclerosis of liver

Central haemorrhagic necrosis of liver

Infarction of liver

Peliosis hepatis
Hepatic angiomatosis

Hepatic veno-occlusive disease

Portal hypertension

Hepatorenal syndrome

Other specified diseases of liver
Focal nodular hyperplasia of liver
Hepatoptosis

Liver disease, unspecified

Intensive-care medicine (critical-care medicine) is a branch of medicine concerned with the diagnosis and management of life threatening conditions requiring sophisticated organ support and invasive monitoring. Sepsis affects approximately 3 in 1000 people a year, and is the second-leading cause of death in intensive care units. Approximately 20-35% of people with severe sepsis and 30-70% of people with septic shock die.

According to the present invention sepsis include the following ICD10 classified sepsis associated diseases:

A40 streptococcal sepsis

A41 other sepsis

### DETAILED DESCRIPTION OF THE INVENTION

CD206

CD206 is also known as MRC1, Macrophage mannose receptor 1, MR, sMR, MMR, C-type lectin domain family 13 member D, C-type lectin domain family 13 member D-like, Macrophage mannose receptor 1-like protein, and 1 CD_antigen=CD206.

CD206/MRC1 exists in humans as a 1456 amino acid long precursor. It consists of an N-terminal signal peptide and contains three domains: an extracellular domain, a transmembrane domain, and a cytoplasmic domain.

MRC1 is expressed as an unprocessed precursor of 1456 amino acids. The molecular weight of the unprocessed precursor is 166 KDa. The protein encoded by the MRC1 gene is classified as a type I transmembrane receptor since the protein C terminus is located on the cytoplasmic side of the membrane.

MRC1 is a membrane receptor containing:
a ricin b-type lectin domain (RICIN), that is a cystein-rich (CysR) domain located at the extreme N terminus and that can bind specific sulphated glycoproteins,
a fibronectin type-II domain (FN2), that is the most conserved of the extracellular domains of the MR family and can bind several forms of collagen,
8 C-type lectin-like domains (CTLDs), that are Ca(2+)-dependent structural motifs.

The fourth of these domains, CTLD4, is the only functional domain. In cooperation with CTLD5, CTLD4 is central to ligand binding by the receptor,
a single transmembrane domain (TM), and
a short cytosolic domain that contains motifs capable of recognizing components of the endocytic pathway.

Probably the MRC1 receptor acts with an alternation between bent and extended conformations, that might serve as a “conformational switch” to regulate ligand binding and receptor activity.

MRC1 interacts with CHEK2 (CHK2 checkpoint homolog—S. pombe) protein.

MRC1 is commonly expressed on macrophages and endothelial cells.

The protein is located in the Plasma membrane and functions by mediating the endocytosis of glycoproteins by macrophages binding both sulfated and non-sulfated polysaccharide chains.

MRC1 acts as a phagocytic receptor binding a range of pathogens, such as bacteria, viruses and fungi, through high-mannose structures that are in their surface.
MRC1 is required for rapid clearance of a subset of mannose-bearing serum glycoproteins that are normally elevated during inflammation.
MRC1 binds and internalises collagen and gelatin in a carbohydrate-independent mechanism.
MRC1 can function as an antigen-acquisition system in a subset of dendritic cells.
MRC1 is implicated in the regulation of macrophage migration during different stages of pathogenesis.
MRC1 has an important role in binding and transmission of HIV-1 by macrophages.

Human MRC1 is an ortholog to murine Mrc1, rat Mrc1, cow LOC787578, chimpanzee MLR1L1, and canine LOC487114. It is a paralog to MRC1L1, CD302, PLA2R1, MRC2.

The extracellular domain of CD206 can be cleaved by metalloproteases (Martinez-Pomares, 2012, J Leukocyte Biol, vol 92 (E-pub doi:10.1189/jlb.0512231); Gazi et al, 2011, J Biol Chem, vol 286:7822-29; Martinez-Pomares et al 1998; Jordens et al 1999, Int Immunol 11,11:1775-1780) to create a soluble CD206 receptor. The soluble CD206 consists of the extracellular domain. The soluble CD206 is capable of binding mannosylated sugars and not sulphated sugars, as multimerisation is required for binding to sulphated sugars (Martinez-Pomares 2012).

Shedding of CD206 by cultured macrophages can be stimulated in vitro by fungal pathogens (Gazi et al, 2011). In vivo, no such stimulation has been reported in the prior art. In contrast, Martinez-Pomares et al 1998 found that the level of soluble CD206 in mouse serum was unaffected by Mycobacterium bovis bacillus, Calmette-Guérin or C. albicans infection or by intraperitoneal treatment with LPS, zymosan or heat-killed C albicans.

CD206 Variants

The present invention involves determining the level of CD206 in a sample from a subject. CD206 is found in a variety of organisms as demonstrated in the sequence alignment of FIG. 7. In each species, the level of CD206 of that species is measured. Within each species there may be variation in the sequence of CD206 to be detected.

Variants can differ from naturally occurring CD206 in amino acid sequence or in ways that do not involve sequence, or in both ways. Variants in amino acid sequence (“sequence variants”) are produced when one or more amino acids in naturally occurring CD206 are substituted with a different natural amino acid, an amino acid derivative or non-native amino acid. Particularly preferred variants include naturally occurring CD206, or biologically active fragments of naturally occurring CD206, whose sequences differ from the wild type sequence by one or more conservative and/or semi-conservative amino acid substitutions, which typically have minimal influence on the secondary and tertiary structure and hydrophobic nature of the protein or peptide. Variants may also have sequences, which differ by one or more non-conservative amino acid substitutions, deletions or insertions, which do not abolish the CD206 biological activity. The alignment in FIG. 7 can be used to predict which amino acid residues can be substituted without substantially affecting the biological activity of the protein.

Substitutions within the following groups (Clustal W, ‘strong’ conservation group) are to be regarded as conservative substitutions within the meaning of the present invention
STA, NEQK, NHQK, NDEQ, QHRK, MILV, MILF, HY, FYW.

Substitutions within the following groups (Clustal W, ‘weak’ conservation group) are to be regarded as semi-conservative substitutions within the meaning of the present invention
CSA, ATV, SAG, STNK, STPA, SGND, SNDEQK, NDEQHK, NEQHRK, VLIM, HFY.

Variants to be detected in one embodiment include proteins and peptides with amino acid sequences having at least 60 percent identity with the soluble part of human, murine, pig, rat or rhesus macaque CD206 (SEQ ID NO: 1, 2, 3, 4 and 5). More preferably the sequence identity is at least 65%, more preferably at least 70%, more preferably at least 75%, more preferably at least 80%, more preferably at least 85%, more preferably at least 90%, more preferably at least 95%, more preferably at least 98

In a preferred embodiment the sequence identity of the variant CD206 is determined with reference to the soluble part of a human CD206 polypeptide (SEQ ID 1).

All aspects of CD206 measurements herein and all detection methods refer to any form of soluble CD206. In a preferred embodiment, the measured CD206 is sCD206.

Associations with Disease States

In one embodiment, sCD206 is used as a sensitive biomarker for liver diseases. In a preferred embodiment, physicians may be enabled to discriminate between high and low risk groups throughout the entire age of a population by obtaining a biological sample from an individual.

Sepsis is a leading cause of death in hospitalized patients, and even though large efforts have been implemented, mortality rates of patients with severe sepsis or septic shock are still unacceptably high.

In another embodiment, sCD206 is used as a sensitive biomarker for sepsis in critically ill patients. In another preferred embodiment, physicians may diagnose risk of sepsis in severe illness by obtaining a biological sample from an individual and assessing the sCD206 level. In another preferred embodiment, physicians may diagnose risk of complications to sepsis (e.g. cardiovascular, or renal, for example, but not restricted to pulmonary complications), by obtaining a biological sample from an individual and assessing the sCD206 level.

In another embodiment, sCD206 is used as a sensitive biomarker for pneumonia and for prognosis of pneumonia patients. In another preferred embodiment, physicians may diagnose disease prognosis in pneumonia by obtaining a biological sample from an individual and assessing the sCD206 level. In another preferred embodiment, physicians may diagnose risk of complications to pneumonia, by obtaining a biological sample from an individual and assessing the sCD206 level.

The present invention relates to the use of sCD206 as a sensitive biomarker for fungal infections. In a preferred embodiment, physicians may discriminate between high and low risk groups throughout the entire age of a population by obtaining a biological sample from an individual and assessing the level of sCD206.

One embodiment relates to the use of sCD206 as a biomarker for fungal infections in critically ill patients. In another preferred embodiment, physicians may diagnose risk of fungal infections in severe illness by obtaining a biological sample from an individual.

Medical Conditions Associated with CD206

The present invention relates to the finding that sCD206 may be used as a biomarker for the presence of an unspecific disease or disorder, as sCD206 is elevated in hospitalised patients and in patients undergoing treatment. Preferably, the disease or disorder is a liver disorder and/or disease. In a more preferred embodiment, the liver disorder is alcoholic liver disease, such as alcoholic fatty liver, for example alcoholic hepatitis, such as alcoholic fibrosis and sclerosis of liver, for example alcoholic cirrhosis of liver, such as alcoholic hepatic failure (acute, chronic, subacute, with or without hepatic coma).

In another preferred embodiment, the liver disorder is toxic liver disease, such as toxic liver disease with cholestatsis (cholestasis with hepatocyte injury, pure cholestasis), for example toxic liver disease with hepatic necrosis (acute hepatic failure, chronic hepatic failure due to drug abuse), such as toxic liver disease with acute or chronic persistent hepatitis, for example toxic liver disease with chronic lobular hepatitis, such as toxic liver disease with chronic active hepatitis, for example toxic liver disease with lupoid hepatitis, such as toxic liver disease with hepatitis, for example toxic liver disease with fibrosis and cirrhosis of liver, such as toxic liver disease with other disorders of liver (focal nodular hyperplasia, hepatic granulomas, peliosis hepatis, veno-occlusive disease of liver).

In yet another preferred embodiment, the liver disorder is hepatic failure (coma NOS, encephalopathy NOS, acute hepatitis, fulminant hepatitis, malignant hepatitis, liver cell necrosis with hepatic failure), such as acute and subacute hepatic failure, for example chronic hepatic failure.

In yet another preferred embodiment, the liver disorder is chronic hepatitis, not elsewhere classified (NEC), such as chronic persistent hepatitis NEC, for example chronic lobular hepatitis NEC, such as chronic active hepatitis (lupoid hepatitis) NEC, for example other chronic hepatitis NEC.

In yet another preferred embodiment, the liver disorder is fibrosis and cirrhosis of liver, such as hepatic fibrosis, for example hepatic sclerosis, such as hepatic fibrosis with hepatic sclerosis, for example primary biliary cirrhosis (chronic nonsuppurative destructive cholangitis), such as secondary biliary cirrhosis, for example unspecified biliary cirrhosis, such as other and unspecified cirrhosis of liver, for example cryptogenic, macronodular, mixed type, portal or postnecrotic cirrhosis of liver. In yet another preferred embodiment, the liver disorder is specified as other inflammatory liver diseases such as abscess of liver (cholangitic, haematogenic, lymphogenic or pylephlebtic hepatic abscess), for example phlebitis (pylephlebitis) of portal vein, such as nonspecific reactive hepatitis, for example granulomatus hepatitis NEC, such as autoimmune hepatitis. As demonstrated in Example 5, sCD206 is elevated in fibrotic or cirrhotic HCV patients compared to non-fibrotic HCV patients.

In yet another preferred embodiment, the liver disorder is specified as other diseases of liver, such as fatty liver NEC, chronic passive congestion of liver (cirrhosis and sclerosis of liver), for example central haemorrhagic necrosis of liver, such as infarction of liver, for example peliosis hepatitis (hepatic angiomatosis), such as hepatic veno-occlusive disease, for example portal hypertension, such as hepatorenal syndrome, for example other specified diseases of liver, including focal nodular hyperplasia of liver and hepatoptosis.

In yet another preferred embodiment, the liver disorder is classified as liver disorders in other diseases, such as cytomegaloviral, herpesviral or toxoplasma hepatitis, for example hepatosplenic schistosomiasis, such as portal hypertension in schistosomiasis, for example syphilitic liver disease, such as hepatic granulomas in berylliosis and sarcoidosis.

sCD206 may be used as a biomarker for the presence of infection. Infection may be caused by bacteria or virus or both.

sCD206 may be used as a biomarker for the presence and severity of pneumonia.

sCD206 may be used as a biomarker for the presence of sepsis. Sepsis is a caused by an overwhelming immune response to infections. The term sepsis is often used interchangeable with septicemia, an infection that gets worse very quickly and is often fatal. Bacterial infections are the most common cause of sepsis. However, sepsis can also be caused by other infections. In a more preferred embodiment, the sepsis is caused by a Gram-positive bacteria, such as Streptococcus pneumonia and Streptococcus pyogenes.

The present invention relates to the finding that sCD206 may be used as a biomarker for the presence of a fungal infection. Fungal infections may be caused by a variety of different fungal species.

In one embodiment, sCD206 may be used as a biomarker for the presence of a number of fungal associated diseases.

In one embodiment the fungal associated diseases are selected from the group consisting of: Candidal endocartis, Candical sepsis, Coccidioidomycosis, Acute pulmonary coccidioidomycosis, Chronic pulmonary coccidioidomycosis, Pulmonary coccidioidomycosis, Cutaneous coccidioidomycosis, Coccidioidomycosis meningitis, Disseminated coccidioidomycosis, Generalized coccidioidomycosis, Other forms of coccidioidomycosis, Histoplasmosis, Acute pulmonary histoplasmosis capsulati, Chronic pulmonary histoplasmosis capsulati, Pulmonary histoplasmosis capsulati, Disseminated histoplasmosis capsulati, Generalized histoplasmosis capsulati, Histoplasmosis capsulati, American histoplasmosis, Histoplasmosis duboisii, African histoplasmosis, Histoplasmosis, Blastomycosis, Acute pulmonary blastomycosis, Chronic pulmonary blastomycosis, Pulmonary blastomycosis, Cutaneous blastomycosis, Disseminated blastomycosis, Generalized blastomycosis, Other forms of blastomycosis, Paracoccidioidomycosis, Pulmonary paracoccidioidomycosis, Disseminated paracoccidioidomycosis, Generalized paracoccidioidomycosis, Other forms of paracoccidioidomycosis, Sporotrichosis, Pulmonary sporotrichosis, Lymphocutaneous sporotrichosis, Disseminated sporotrichosis, Generalized sporotrichosis, Other forms of sporotrichosis, Chromomycosis and phaeomycotic abscess, Cutaneous chromomycosis, Dermatitis verrucosa, Phaeomycotic brain abscess, Cerebral chromomycosis, Subcutaneous phaeomycotic abscess and cyst, Other forms of chromomycosis, Aspergillosis, Invasive pulmonary aspergillosis, Other pulmonary aspergillosis, Tonsillar aspergillosis, Disseminated aspergillosis, Generalized aspergillosis, Other forms of aspergillosis, Cryptococcosis, Pulmonary cryptococcosis, Cerebral cryptococcosis, Cryptococcal meningitis, Cryptococcosis meningocerebralis, Cutaneous cryptococcosis, Osseous cryptococcosis, Disseminated cryptococcosis, Generalized cryptococcosis, Other forms of cryptococcosis, Zygomycosis, Pulmonary mucormycosis, Rhinocerebral mucormycosis, Gastrointestinal mucormycosis, Cutaneous mucormycosis, Subcutaneous mucormycosis, Disseminated mucormycosis, Generalized mucormycosis, Mucormycosis unspecified, Other zygomycoses, Entomophthoromycosis, Zygomycosis unspecified, Phycomycosis NOS, Mycetoma, Eumycetoma, Madura foot mycotic, Maduromycosis, Actinomycetoma, Mycetoma unspecified, Madura foot NOS, Lobomycosis, Keloidal blastomycosis, Lobo disease, Rhinosporidiosis, Allescheriasis, Infection due to Pseudallescheria boydii, Geotrichosis, Geotrichum stomatitis, Penicillosis, Opportunistic mycoses, Other specified mycoses, and Adiaspiromycosis.

Sampling of CD206

In a preferred embodiment, the level of CD206 will be obtained from a biological sample, such as serum, for example plasma, such as whole blood, for example saliva, such as urine, for example lymph, such as a biopsy, for example semen, such as faeces, for example tears, such as sweat, for example milk, such as cerebrospinal fluid, for example ascites fluid, such as for example synovial fluid. Preferably the sample is blood, plasma or serum. More preferably the sample is plasma or serum.

Methods for Determining CD206

Point of Care test preferably relies on a lateral flow test based on an immunological principle. Lateral flow tests are also known as lateral flow immunochromatographic assays and are simple devices intended to detect the presence (or absence) of a target analyte in a sample. Often produced in a dipstick format, a lateral flow test is a form of immunoassay in which the test sample flows along a solid substrate, preferably via capillary action. After the sample is applied to the test it preferably encounters a coloured reagent which mixes with the sample and transits the substrate encountering lines or zones which have been pretreated with an antibody or antigen. Depending upon the analytes present in the sample the coloured reagent can become bound at the test line or zone. Semi-quantitative lateral flow tests can operate as either competitive or sandwich assays.

In a preferred embodiment, the sample is mixed with CD206 antibody-coated microparticles with a resulting change in the turbidity of the sample. The turbidity change may then be correlated with the amount of CD206 in the sample when compared with a reference sample.

In another preferred embodiment, the level of CD206 is detected by nephelometry where an antibody and the antigen are mixed in concentrations such that only small aggregates are formed. These aggregates will scatter light (usually a laser) passed through it rather than simply absorbing it. The fraction of scattered light is determined by collecting the light at an angle where it is measured and compared to the fraction of scattered light from known mixtures. Scattered light from the sample is determined by using a standard curve.

In another preferred embodiment, the sample moves from the application site where it, for example, is mixed with antibody-coated nanoparticles in lateral flow/diffusion through a (e.g. nitrocellulose-) membrane. At one point on the way another CD206 antibody is fixed in the membrane making the CD206-primary antibody complex to halt. The nano-particle (preferably colloidal gold/dyed latex) will give a visual line. In another embodiment, the sample is applied through a (e.g. nitrocellulose-) membrane coated with a primary CD206 antibody. The sample CD206 is then recognised and bound by the primary CD206 antibody. The immobilised CD206 on the membrane may then be recognised by (preferably colloidal gold/dyed latex) particles conjugated with another CD206 antibody, and the complex will develop a colour reaction, which intensity corresponds to the amount of CD206 in the sample.

For large-scale detection and more precise quantitative measurement of CD206 in a sample, several methods may be applied.

In another preferred embodiment, the level of CD206 is detected by radioimmunoassay (RIA). RIA is a very sensitive technique used to measure concentrations of antigens without the need to use a bioassay. To perform a radioimmunoassay, a known quantity of an antigen is made radioactive, frequently by labeling it with gamma-radioactive isotopes of iodine attached to tyrosine. This radio labeled antigen is then mixed with a known amount of antibody for that antigen, and as a result, the two chemically bind to one another. Then, a sample of serum from a patient containing an unknown quantity of that same antigen is added. This causes the unlabeled (or “cold”) antigen from the serum to compete with the radio labeled antigen for antibody binding sites. As the concentration of “cold” antigen is increased, more of it binds to the antibody, displacing the radio labeled variant, and reducing the ratio of antibody-bound radio labeled antigen to free radio labeled antigen. The bound antigens are then separated from the unbound ones, and the radioactivity of the free antigen remaining in the supernatant is measured. Using known standards, a binding curve can then be generated which allows the amount of antigen in the patient's serum to be derived. In this assay, the binding between antibody and antigen may be substituted by any protein-protein or protein-peptide interaction, such as ligand-receptor interaction, for example CD206-haemoglobin or CD206-haemoglobin/haptoglobin binding.

In a preferred embodiment, the level of CD206 is detected by enzyme-linked immunosorbent assay (ELISA). ELISA is a quantitative technique used to detect the presence of protein, or any other antigen, in a sample. In ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Several types of ELISA exist:

Indirect ELISA

Sandwich ELISA

Competitive ELISA

Reverse ELISA

Other immuno-based assays may also be used to detect CD206 in a sample, such as chemiluminescent immunometric assays and Dissociation-Enhanced Lanthinide Immunoassays.

In a preferred embodiment, the level of CD206 is detected by chromatography-based methods, more specifically liquid chromatography. Therefore, in a more preferred embodiment, the level of CD206 is detected by affinity chromatography which is based on selective non-covalent interaction between an analyte and specific molecules. In another preferred embodiment, the level of CD206 is detected by ion exchange chromatography which uses ion exchange mechanisms to separate analytes. Ion exchange chromatography uses a charged stationary phase to separate charged compounds. In conventional methods the stationary phase is an ion exchange resin that carries charged functional groups which interact with oppositely charged groups of the compound to be retained.

In yet another preferred embodiment, the level of CD206 is detected by size exclusion chromatography (SEC) which is also known as gel permeation chromatography (GPC) or gel filtration chromatography. SEC is used to separate molecules according to their size (or more accurately according to their hydrodynamic diameter or hydrodynamic volume). Smaller molecules are able to enter the pores of the media and, therefore, take longer to elute, whereas larger molecules are excluded from the pores and elute faster.

In yet another preferred embodiment, the level of CD206 is detected by reversed-phase chromatography which is an elution procedure in which the mobile phase is significantly more polar than the stationary phase. Hence, polar compounds are eluted first while non-polar compounds are retained.

In a preferred embodiment, the level of CD206 is detected by electrophoresis. Electrophoresis utilizes the motion of dispersed particles relative to a fluid under the influence of an electric field. Particles then move with a speed according to their relative charge. More specifically, the following electrophoretic methods may be used for detection of CD206:

Sodium dodecyl sulfate polyacrylamide gel electrophoresis (SDS-PAGE).

Rocket immunoelectrophoresis.

Affinity immunoelectrophoresis.

Isoelectric focusing.

In a preferred embodiment, the level of CD206 is detected by flow cytometry. In flow cytometry a beam of light of a single wavelength is directed onto a hydrodynamically-focused stream of fluid. A number of detectors (some fluorescent) are aimed at the point where the stream passes through the light beam: one in line with the light beam and several detectors perpendicular to it. Each suspended particle from 0.2 to 150 micrometers passing through the beam scatters the light in some way, and fluorescent chemicals found in the particle or attached to the particle may be excited into emitting light at a longer wavelength than the light source. This combination of scattered and fluorescent light is picked up by the detectors, and, by analysing fluctuations in brightness at each detector, it is then possible to derive various types of information about the physical and chemical structure of each individual particle.

In a preferred embodiment, the level of CD206 is detected by Luminex technology, which is based on a technique where microspheres are coated with reagents specific to capture a specific antigen from a sample.

In a preferred embodiment, the level of CD206 is detected by mass spectrometry (MS). MS is an analytical technique for the determination of the elemental composition of a sample or molecule. It is also used for elucidating the chemical structures of molecules, such as proteins and other chemical compounds. The MS principle consists of ionizing chemical compounds to generate charged molecules or molecule fragments and measurement of their mass-to-charge ratios.

Determination of Cut-Off Value

In order to evaluate the level of CD206 as measured by any of the methods mentioned herein above, the establishment of a cut-off value may be beneficial.

The cut-off level as herein defined may also be regarded a control level to which the sCD206 level of a sample can be compared.

In one embodiment a cut-off value may be determined by measuring the level of CD206 in a given healthy subject several times over an extended period of time. In that way it is possible to determine the average normal level of CD206 in said subject.

In another embodiment a cut-off value may be determined for a given sick subject by measuring the level of CD206 in a sample from said given subject, provided that said sample has been taken prior to the onset of disease. The level measured in the sample taken when the subject was healthy is then used as the cut-off value for when the subject may be categorized as being healthy again after the disease has been treated.

In yet another embodiment a cut-off value may be determined by a mathematical value based on an average of measurement made in a large group of subjects. Said subjects may be grouped according to their age, gender, race and population group. Preferably said subjects are healthy subjects, i.e. without any known medical condition.

The skilled person will know that in biological systems some variation around an average should be expected and will still be accepted as a normal value. In one embodiment a value will be accepted as normal if it does not deviate significantly from the average value determined to be the cut-off value.

A low level of CD206 is indicative of a subject being healthy. Accordingly it is expected that the measured CD206 level in a subject suffering from a disorder and/or disease should fall after the subject has received treatment for the disorder and/or disease. Treatment may continue until the CD206 level is below the determined cut-off value. Once the CD206 level is below the determined cut-off value the subject will be evaluated as being healthy again.

A cut-off value according is preferably a cut-off value where the biomarker is both sensitive and specific. It is desired that the correlation between sensitivity and specificity is maximized.

One way of determining whether a given cut-off value is sensitive and specific is by plotting the data obtained in a Receiver Operating Characteristic (ROC) curve (see FIG. 9).

Said curve is created by plotting the fraction of true positives out of the positives (TPR=true positive rate) vs. the fraction of false positives out of the negatives (FPR=false positive rate), at various threshold settings. TPR is also known as sensitivity, and FPR is one minus the specificity or true negative rate.

An ideal cut-off value would discriminate faultlessly between patient with or without a disease or disorder, such as with and without liver disorder and/or sepsis. Where the given cut-off value identifies only the true positives, it will have a sensitivity of 100% or 1.00. Where the test identifies only the true negatives, it will have a specificity of 100% or 1.00.

When determining where to set the cut-off value, the skilled person would have to make a balance between the need to identify all the patients suffering from the disease or disorder, such as from sepsis and/or a liver disorder, from the patients not suffering from said diseases. The effect of raising or lowering the cut-off value will have well-defined and predictable impact on the sensitivity and specificity. The skilled person will know that there is some overlap between “disease absent” and “disease present” patient populations.

The level of sCD206 may be measured on a variety of samples as described herein above.

A cutoff level may be an average of measurement made in a large group of healthy subjects, optionally grouped according to age, gender, race, or population group. In absolute numbers, a general cutoff level may be at least 0.25 mg/L sCD206, such as at least 0.3 mg/L, for example at least 0.35 mg/L, such as at least 0.4 mg/L, for example at least 0.43 mg/L, such as at least 0.5 mg/L. These absolute values have been determined using the ELISA-assay described in the examples. Using this assay, the level of sMR in NFKK reference-serum X has been found to be 0.243 mg/L (SD=0.030). NFKK-reference serum X, which is commercially available from NOBIDA, Nordic Reference Interval Project Bio-bank and Database (Scand J Clin Lab Invest. 2004; 64(4):431-8. Nordic Reference Interval Project Bio-bank and Database (NOBIDA): a source for future estimation and retrospective evaluation of reference intervals. Rustad P, Simonsson P, Felding P, Pedersen M.).

It is conceivable that other analytical methods will yield different concentrations in absolute numbers. The cutoff values provided in the present application can be converted to cutoff values for other analytical methods. If the concentration of sMR in NFKK-X using a different analytical method is found to be “C mg/L”, then all reported cut-off values for sMR using the methods herein described should be multiplied by the constant “C/0.243”.

In general the distinction between healthy and unhealthy individuals may involve comparing a value to a cutoff value, wherein said cutoff level is at least 1.5×a median level of sCD206 for healthy individuals, such as 1.75, for example 2.0, such as 2.25, for example 2.5, such as 2.75, for example 3.0, such as 3.25, for example 3.5.

In one embodiment the cut-off value when determining whether or not a patient is suffering from sepsis or not is 0.7 mg/L of sCD206, for example the cut-off value is 0.68 mg/L of sCD206, for example the cut-off value is 0.65 mg/L of sCD206, for example the cut-off value is 0.63 mg/L of sCD206, for example the cut-off value is 0.61 mg/L of sCD206.

In another embodiment the cut-off value when determining whether or not a patient is suffering from sepsis or not is 0.58 mg/L of sCD206, for example the cut-off value is 0.56 mg/L of sCD206, for example the cut-off value is 0.54 mg/L of sCD206, for example the cut-off value is 0.52 mg/L of sCD206, for example the cut-off value is 0.5 mg/L of sCD206, for example the cut-off value is 0.48 mg/L of sCD206, for example the cut-off value is 0.45 mg/l of sCD206, for example the cut-off value is 0.43 mg/L of sCD206.

In another embodiment the cut-off value when determining whether or not a patient is suffering from sepsis or not is at least 0.4 mg/L, for example at least 0.43 mg/L, such as at least 0.5 mg/L, for example at least 0.6 mg/L.

In one embodiment, the cut-off value when determining the likelihood of survival of a patient admitted to the ICU is 0.4 mg/L of sCD206, for example the cut-off value is 0.45 mg/L of sCD206, for example the cut-off value is 0.5 mg/L of sCD206, for example the cut-off value is 0.55 mg/L of sCD206, for example the cut-off value is 0.6 mg/L of sCD206, for example the cut-off value is 0.65 mg/L of sCD206, for example the cut-off value is 0.7 mg/L of sCD206, for example the cut-off value is 0.75 mg/L of sCD206, for example the cut-off value is 0.8 mg/L of sCD206, for example the cut-off value is 0.85 mg/L of sCD206, for example the cut-off value is 0.9 mg/L of sCD206, for example the cut-off value is 0.95 mg/L of sCD206, for example the cut-off value is 1.0 mg/L of sCD206. These cut-off values may also be used to discriminate between survivors and non-survivors among pneumonia patients.

In another embodiment the cut-off value when determining the likelihood of survival of a patient admitted to the ICU is at least 0.4 mg/L, for example at least 0.5 mg/L, such as at least 0.6 mg/L, for example at least 0.7 mg/L, such as at least 0.8 mg/L, for example at least 0.9 mg/L, such as at least 1.0 mg/L.

In one embodiment the cut-off value may be related to either the 97.5^{th} percentile or the median value of a group of healthy individuals. The advantage of not using absolute values is that absolute values may vary depending on the method used to determine soluble CD206.

In one embodiment the cut-off value is related to the percentile groups described herein below and is defined as a value multiplied by (herein below “multiplied by” is denoted with an x) the value determined for the 97.5^{th} percentile of healthy individuals. In such an embodiment the cut-off value when determining whether or not a patient is suffering from sepsis is 1.3×the 97.5^{th} percentile value of healthy individuals, for example the cut-off value is 1.35×the 97.5^{th} percentile value of healthy individuals, for example the cut-off value is 1.4×the 97.5^{th} percentile value of healthy individuals, for example the cut-off value is 1.45×the 97.5^{th} percentile value of healthy individuals, for example the cut-off value is 1.5×the 97.5^{th} percentile value of healthy individuals.

In another embodiment the cut-off value when determining the likelihood of survival of a patient admitted to the ICU is 0.9×97.5^{th} percentile value of healthy individuals, for example the cut-off value is 1.0×97.5^{th} percentile value of healthy individuals, for example the cut-off value is 1.1×97.5^{th} percentile value of healthy individuals, for example the cut-off value is 1.2×97.5^{th} percentile value of healthy individuals, for example the cut-off value is 1.3×97.5^{th} percentile value of healthy individuals, for example the cut-off value is 1.4×97.5^{th} percentile value of healthy individuals, for example the cut-off value is 1.5×97.5^{th} percentile value of healthy individuals, for example the cut-off value is 1.6×97.5^{th} percentile value of healthy individuals, for example the cut-off value is 1.7×97.5^{th} percentile value of healthy individuals, for example the cut-off value is 1.8×97.5^{th} percentile value of healthy individuals, for example the cut-off value is 1.9×97.5^{th} percentile value of healthy individuals, for example the cut-off value is 2.0×97.5^{th} percentile value of healthy individuals, for example the cut-off value is 2.1×97.5^{th} percentile value of healthy individuals, for example the cut-off value is 2.2×97.5^{th} percentile value of healthy individuals, for example the cut-off value is 2.3×97.5^{th} percentile value of healthy individuals.

In one embodiment the cut-off value is related to the median value determined for healthy individuals and is defined as a value multiplied by (herein below “multiplied by” is denoted with an x) the median value determined for healthy individuals.

In such an embodiment the cut-off value when determining whether or not a patient is suffering from sepsis is 2.0×the median value of healthy individuals, for example the cut-off value is 2.05×the median value for healthy individuals, for example the cut-off value is 2.1×the median value of healthy individuals, for example the cut-off value is 2.15×the median value of healthy individuals, for example the cut-off value is 2.2×the median value of healthy individuals, for example the cut-off value is 2.25×the median value for healthy individuals, for example the cut-off value is 2.3×the median value for healthy individuals.

In another embodiment the cut-off value when determining the likelihood of survival of a patient admitted to the ICU is 1.40×the median value of healthy individuals, for example the cut-off value is 1.50×the median value for healthy individuals, for example the cut-off value is 1.60×the median value for healthy individuals, for example the cut-off value is 1.70×the median value for healthy individuals, for example the cut-off value is 1.80×the median value for healthy individuals, for example the cut-off value is 1.90×the median value for healthy individuals, for example the cut-off value is 2.00×the median value for healthy individuals, for example the cut-off value is 2.10×the median value for healthy individuals, for example the cut-off value is 2.20×the median value for healthy individuals, for example the cut-off value is 2.30×the median value for healthy individuals, for example the cut-off value is 2.40×the median value for healthy individuals, for example the cut-off value is 2.50×the median value for healthy individuals, for example the cut-off value is 2.60×the median value for healthy individuals, for example the cut-off value is 2.70×the median value for healthy individuals, for example the cut-off value is 2.80×the median value for healthy individuals, for example the cut-off value is 2.90×the median value for healthy individuals, for example the cut-off value is 3.00×the median value for healthy individuals, for example the cut-off value is 3.10×the median value for healthy individuals, for example the cut-off value is 3.20×the median value for healthy individuals, for example the cut-off value is 3.30×the median value for healthy individuals, for example the cut-off value is 3.40×the median value for healthy individuals, for example the cut-off value is 3.50×the median value for healthy individuals, for example the cut-off value is 3.60×the median value for healthy individuals.

Alternatively the average value for healthy individuals may be used instead of the median value.

Percentile Groups

In another embodiment percentiles of sCD206 value are determined in a larger population over an extended period of time. The level of sCD206 found in a larger population can then be divided into percentiles. E.g. the 20th percentile is the value (or score) below which 20 percent of the observations are found. The percentiles can be used to determine a reference level of sCD206 (percentiles from e.g. 0-33%) and to determine “high level” of sCD206 (e.g. 90^{th} percentile or lower as described herein below).

Therefore, in one embodiment a high level of sCD206 comprises a value higher than the 60^{th} percentile, for example higher than the 65^{th} percentile, such as higher than the 67^{th} percentile, for example higher than the 70^{th} percentile, such as higher than the 75^{th} percentile, for example higher than the 80^{th} percentile, such as higher than the 85^{th} percentile, for example higher than the 90^{th} percentile, such as higher than the 95^{th} percentile, for example higher than the 97^{th} percentile.

A high value may also be determined with relatively to a percentile. According to such methods, a high level of sCD206 comprises a value higher than 0.9×97.5^{th} percentile, such as higher than 1.0×97.5^{th} percentile, for example 1.1×97.5^{th} percentile, such as 1.2×97.5^{th} percentile, for example 1.3×97.5^{th} percentile, such as 1.4×97.5^{th} percentile, for example 1.5×97.5^{th} percentile, such as 1.6×97.5^{th} percentile, for example 1.7×97.5^{th} percentile, such as 1.8×97.5^{th} percentile, for example 1.9×97.5^{th} percentile, such as 2.0×97.5^{th} percentile, for example 2.1×97.5^{th} percentile, such as 2.2×97.5^{th} percentile, for example 2.3×97.5^{th} percentile, such as 2.4×97.5^{th} percentile, for example 2.5×97.5^{th} percentile.

Percentiles and percentiles multiplied by a factor may also be regarded as cutoff values.

In another preferred embodiment, said percentiles are determined for a subset of individuals, said individuals having the same gender or race, or belonging to a group based on age, BMI, smoking habit, occupation, physical inactivity, hip circumference, waist circumference, systolic and/or diastolic blood pressure, alcohol consumption, a combination of any subset of these, or other risk factor. In a more preferred embodiment, said percentiles are determined for a subset of individuals, said individuals having the same gender and belonging to the same age interval, said interval being 5 years, 10 years, 15 years, 20 years or said interval being 25 years.

Said percentiles are based on multiple factors, among those CD206 levels, gender and age. When classified into 10-year age intervals, it is possible to derive absolute cut-off values, above which an individual is at risk of contracting said disorders.

The risk of suffering from liver disease or sepsis among said individuals may be determined from which percentile an individual belongs to. The risk of suffering from said disease may be calculated by comparing to a reference group.

Divided into percentiles based on CD206 levels, age and gender, a preferred reference group is the group with the lowest risk of suffering from a disease or disorder.

Additional Assessments

Several biochemical parameters are known to be associated with liver disease or sepsis. A normal procedure in the clinical laboratory may be to confirm positive and negative findings obtained by assessing one biochemical marker (of for example a disorder) by assessing the presence of other, independent biochemical markers with similar clinical indications.

In another preferred embodiment the use of CD206 as a biomarker for said diseases may be supported by assessing measures such as Apache-II score, Model for End-Stage Liver Disease (MELD score), Sequential Organ Failure Assessment score (SOFA score), or the Ostrosky-Zeichner prediction rule, or other, related biochemical markers obtained from a group of, but not limited to, Creatinine, Lactate, blood glucose, CRP, Fibrinogen, alpha1-antitrypsin, ALAT, ASAT, gammaGT, alkaline phosphatise, coagulation factors, Thrombocyte counts, lactate dehydrogenase, homocysteine, and bilirubine.

Treatment of Subjects with Increased CD206

One great asset of a biomarker is that it paves the way for an individual to take actions aimed at treating a certain disease at an early time point before overt signs of said disease develop. Said actions may include (in the ICU) an intensified monitoring, antibiotic treatment, organ-support (pressors, ventilation, dialysis) and in the case of liver disease in the search for viral disease, and altered daily routines, such as increased physical activity and a healthier diet, such as reduced consumption of fat, sugar and alcohol. Moreover, a number of compounds are undergoing clinical trials to investigate their effect on lowering low-grade systemic inflammation or subclinical inflammation. Examples of such drugs include but are not limited to:

Coffee, Glucose-dependent insulinotropic polypeptide (GIP), nicotinic acid, pioglitazone, ramipril, curcumin, fructanes, acarbose, vitamin D, butyrate, thiazolidinediones, mesalazine, salsalate, advair, flovent, atenolol, ramipril, metformin Glucagon-like peptide-1 agonists, and resveratrol.

Resveratrol (3,5,4′-trihydroxystilbene) is a polyphenolic phytoalexin. It is a stilbenoid, a derivate of stilbene, and is produced in plants with the help of the enzyme stilbene synthase. It exists as two structural isomers: cis-(Z) and trans-(E), with the trans-isomer shown in the top image. The trans-form can undergo isomerisation to the cis-form when heated or exposed to ultraviolet irradiation. Resveratrol is a polyphenol found in red wine.

The level of CD206 may be measured several times during treatment in order to evaluate the treatment regime. The level of CD206 will then inform the physician of whether or not a certain treatment is useful and providing an improvement in the general health of the subject or whether the treatment should be altered. If the level of sCD206 increases during treatment the physician will know that the treatment eventually should be stopped. A new treatment with another pharmaceutical compound may be administered subsequently.

Alterations in treatment could in one embodiment be a change in dosage of a given pharmaceutical compound or it may be a partial or complete change in medicament.

### EXAMPLES

### Example 1

### Sequence Listing

SEQ ID No. 1, CD206, Uniprot acc no. P22897

Signal peptide: 1-19

Extracellular domain: 19-1389

Transmembrane: 1390-1410

Cytoplasmic: 1411-1456

>gi|4505245|ref|NP_002429.1| macrophage mannose receptor 1 precursor
[Homo sapiens]
MRLPLLLVFASVIPGAVLLLDTRQFLIYNEDHKRCVDAVSPSAVQTAACNQDAESQKFRWVSESQIMSVA
FKLCLGVPSKTDWVAITLYACDSKSEFQKWECKNDTLLGIKGEDLFFNYGNRQEKNIMLYKGSGLWSRWK
IYGTTDNLCSRGYEAMYTLLGNANGATCAFPFKFENKWYADCTSAGRSDGWLWCGTTTDYDTDKLFGYCP
LKFEGSESLWNKDPLTSVSYQINSKSALTWHQARKSCQQQNAELLSITEIHEQTYLTGLTSSLTSGLWIG
LNSLSFNSGWQWSDRSPFRYLNWLPGSPSAEPGKSCVSLNPGKNAKWENLECVQKLGYICKKGNTTLNSF
VIPSESDVPTHCPSQWWPYAGHCYKIHRDEKKIQRDALTTCRKEGGDLTSIHTIEELDFIISQLGYEPND
ELWIGLNDIKIQMYFEWSDGTPVTFTKWLRGEPSHENNRQEDCVVMKGKDGYWADRGCEWPLGYICKMKS
RSQGPEIVEVEKGCRKGWKKHHFYCYMIGHTLSTFAEANQTCNNENAYLTTIEDRYEQAFLTSFVGLRPE
KYFWTGLSDIQTKGTFQWTIEEEVRFTHWNSDMPGRKPGCVAMRTGIAGGLWDVLKCDEKAKFVCKHWAE
GVTHPPKPTTTPEPKCPEDWGASSRTSLCFKLYAKGKHEKKTWFESRDFCRALGGDLASINNKEEQQTIW
RLITASGSYHKLFWLGLTYGSPSEGFTWSDGSPVSYENWAYGEPNNYQNVEYCGELKGDPTMSWNDINCE
HLNNWICQIQKGQTPKPEPTPAPQDNPPVTEDGWVIYKDYQYYFSKEKETMDNARAFCKRNFGDLVSIQS
ESEKKFLWKYVNRNDAQSAYFIGLLISLDKKFAWMDGSKVDYVSWATGEPNFANEDENCVTMYSNSGFWN
DINCGYPNAFICQRHNSSINATTVMPTMPSVPSGCKEGWNFYSNKCFKIFGFMEEERKNWQEARKACIGF
GGNLVSIQNEKEQAFLTYHMKDSTFSAWTGLNDVNSEHTFLWTDGRGVHYTNWGKGYPGGRRSSLSYEDA
DCVVIIGGASNEAGKWMDDTCDSKRGYICQTRSDPSLTNPPATIQTDGFVKYGKSSYSLMRQKFQWHEAE
TYCKLHNSLIASILDPYSNAFAWLQMETSNERVWIALNSNLTDNQYTWTDKWRVRYTNWAADEPKLKSAC
VYLDLDGYWKTAHCNESFYFLCKRSDEIPATEPPQLPGRCPESDHTAWIPFHGHCYYIESSYTRNWGQAS
LECLRMGSSLVSIESAAESSFLSYRVEPLKSKTNFWIGLFRNVEGTWLWINNSPVSFVNWNTGDPSGERN
DCVALHASSGFWSNIHCSSYKGYICKRPKIIDAKPTHELLTTKADTRKMDPSKPSSNVAGVVIIVILLIL
TGAGLAAYFFYKKRRVHLPQEGAFENTLYFNSQSSPGTSDMKDLVGNIEQNEHSVI
>gi|224967062|ref|NP_032651.2| macrophage mannose receptor 1 precursor
[Mus musculus]
SEQ ID NO: 2
MRLLLLLAFISVIPVSVQLLDARQFLIYNEDHKRCVDALSAISVQTATCNPEAESQKFRWVSDSQIMSVA
FKLCLGVPSKTDWASVTLYACDSKSEYQKWECKNDTLFGIKGTELYFNYGNRQEKNIKLYKGSGLWSRWK
VYGTTDDLCSRGYEAMYSLLGNANGAVCAFPFKFENKWYADCTSAGRSDGWLWCGTTTDYDKDKLFGFCP
LHFEGSERLWNKDPLTGILYQINSKSALTWHQARASCKQQNADLLSVTEIHEQMYLTGLTSSLSSGLWIG
LNSLSVRSGWQWAGGSPFRYLNWLPGSPSSEPGKSCVSLNPGKNAKWENLECVQKLGYICKKGNNTLNPF
IIPSASDVPTGCPNQWWPYAGHCYRIHREEKKIQKYALQACRKEGGDLASIHSIEEFDFIFSQLGYEPND
ELWIGLNDIKIQMYFEWSDGTPVTFTKWLPGEPSHENNRQEDCVVMKGKDGYWADRACEQPLGYICKMVS
QSHAVVPEGADKGCRKGWKRHGFYCYLIGSTLSTFTDANHTCTNEKAYLTTVEDRYEQAFLTSLVGLRPE
KYFWTGLSDVQNKGTFRWTVDEQVQFTHWNADMPGRKAGCVAMKTGVAGGLWDVLSCEEKAKFVCKHWAE
GVTRPPEPTTTPEPKCPENWGTTSKTSMCFKLYAKGKHEKKTWFESRDFCKAIGGELASIKSKDEQQVIW
RLITSSGSYHELFWLGLTYGSPSEGFTWSDGSPVSYENWAYGEPNNYQNVEYCGELKGDPGMSWNDINCE
HLNNWICQIQKGKTLLPEPTPAPQDNPPVTADGWVIYKDYQYYFSKEKETMDNARAFCKKNFGDLATIKS
ESEKKFLWKYINKNGGQSPYFIGMLISMDKKFIWMDGSKVDEVAWATGEPNFANDDENCVTMYTNSGFWN
DINCGYPNNFICQRHNSSINATAMPTTPTTPGGCKEGWHLYKNKCFKIFGFANEEKKSWQDARQACKGLK
GNLVSIENAQEQAFVTYHMRDSTFNAWTGLNDINAEHMFLWTAGQGVHYTNWGKGYPGGRRSSLSYEDAD
CVVVIGGNSREAGTWMDDTCDSKQGYICQTQTDPSLPVSPTTTPKDGFVTYGKSSYSLMKLKLPWHEAET
YCKDHTSLLASILDPYSNAFAWMKMHPFNVPIWIALNSNLTNNEYTWTDRWRVRYTNWGADEPKLKSACV
YMDVDGYWRTSYCNESFYFLCKKSDEIPATEPPQLPGKCPESEQTAWIPFYGHCYYFESSFTRSWGQASL
ECLRMGASLVSIETAAESSFLSYRVEPLKSKTNFWIGMFRNVEGKWLWLNDNPVSFVNWKTGDPSGERND
CVVLASSSGLWNNIHCSSYKGFICKMPKIIDPVTTHSSITTKADQRKMDPQPKGSSKAAGVVTVVLLIVI
GAGVAAYFFYKKRHALHIPQEATFENTLYFNSNLSPGTSDTKDLMGNIEQNEHAII
>gi|157822935|ref|NP_001099593.1| macrophage mannose receptor 1 precursor
[Rattus norvegicus]
SEQ ID NO: 3
MEHTLWAMRLPLLLAFISVIPVAVQLLDSRQFLIYNEDHKRCVDALSAISVQTATCNPEAESQKFRWVSE
SQIMSVAFKLCLGVPSKTDWASVTLYACDSKSEFQKWECKNDTLFGIKGTELYFNYGNRQEKNIKLYKGS
GLWSRWKVYGTTDDLCSRGYEAMYSLLGNANGAVCAFPFKFENKWYADCTSAGRSDGWLWCGTTTDYDTD
KLFGFCPLQFEGSKRLWNTDPLTGILYQINSKSALTWHQARASCKQQNAELLSVTEIHEQMYLTGLTSSL
TSGLWIGLNSLSLSTGWQWAGGSPFRYLNWLPGSPSSEPGKSCVSLNPGKNAKWENLECVQKLGYICKKG
NNTLNPFIIPSESDVPTACPNQWWPYAGHCYRIYREEKKIQKYALQACRKEGGDLASIHSIEEFDFIFSQ
LGYEPNDELWIGLNDIKIQMYFEWSDGTPVTFTKWLPGEPSHENNRQEDCVVMKGKDGYWADRACEQPLG
YICKMVSQIHTVIPEGAEKGCRKGWKRHGFYCYLIGSTLSTFADANQTCTNEKAYLTTVEDRYEQAFLTS
LVGLRPEKYFWTGLSDVQNKGTFRWTVDEQVQFTHWNADMPGRKAGCVAMKTGVAGGLWDVLSCEEKAKF
VCKHWAEGVTRPPEPTTTPEPKCPEDWGTTSKTSMCFKLYAKGKHEKKTWFESRDFCKAIGGELASIKSK
DEQQVIWRLITSSGSYHELFWLGLTYGSPSEGFTWSDGSPVSYENWAYGEPNNYQNVEYCGELKGDPGMS
WNDINCEHLNNWICQIQKGKTLLPEPTPAPQDNPPVTADGWVIYKDYQYYFSKEKETMDNARAFCKKNFG
DLATIKSESEKKFLWKYINKNGGQSPYFIGLLISLDKKFIWMDGSKVDFVAWATGEPNFANDDENCVTMY
TNSGFWNDINCGYPNNFICQRHNSSINATAMPTTPPTPGGCKEGWHLYNNKCFKIFGFAEEEKKTWKEAR
KACIGLKGNLVSIENAKEQAFVTYHMRDSTFNAWTGLNDVNSEHTFLWTDGRGVHYTNWGKGYPGGRRSS
LSYEDVDCVVVIGGNSREAGTWMDSTCDSKQGYICQTQTDPSVPISPTTAPKDGFVKYGKSSYALMKLKS
PWHEAEKYCKDRTSLLASILDPYSNAFAWMKMHPFNVPIWIALNSNLTNNEYTWTDKWRVRYTNWGTDEP
KLKSACVYMDVDGYWKTSYCNESFYFLCKKSDEIPATEPPQLPGKCPESEQTAWIPFHGHCYYIESSFTR
SWGQASLECLRMGASLVSIETAAESSFLSYRVEPLKSKTNFWIGMFRNVEGKWLWLNDNPVSFVNWKTGD
PSGERNDCVVLSSSSGLWNNIHCTSYKGFICKMPKIIDPVTTHSSITTKADQRKMDPQPKGSSKAAGVVI
VVLLIVIGAGVAAYFFYKKRRVLHIPQEATFENTLYFNSNPSPGTSDTKDLVGNIEQNEHAVI
>gi|363814526|ref|NP_001242898.1| macrophage mannose receptor 1 precursor
[Sus scrofa]
SEQ ID NO: 4
MRLSPCLAFLSFLPVALQLLDTRQFLIYNEDHKRCVEALSPSSVQTAVCNQDNEAQKFRWVSESQIMSVA
FKLCLGVPSKTDWVPVTLYACDSKSEFQKWECRNDTLLGIKGEDIFFNYGNRQEKNIMLYKGSGLWSRWK
VYGTTDDLCSRGYEAMYTLLGNSNGATCAFPFKFENKWYADCTTAGRSDGWLWCGTTTDYDTDKLFGYCP
LKFEGIERLWNKDPLTSISYQINSKSALTWHQARKSCQQQNAELLSITEIHEQTYLTGLTSSLTSGLWIG
LNSLSFNSGWQWSGGSPFRYLNWLPGSPSAEPGKSCVSLNPGKNAKWENLQCVQKLGYICKKGNTTLNSF
VIPSESDVPTSCPSQWWPYAGHCYKIYREEKKIQRDALTACRKEGGDLASIHSIEEFDFIISQLGYEPND
ELWIGLNDIKIQMYFEWSDGTPVTFTKWLPGEPSHENNRQEDCAVMKGKDGYWADRACERPLGYICKMKS
QAQTPGRVEVETGCRKGWKRHGFYCYLIGHTLSTFAEANQTCENEKAYLTTVEDRYEQAFLTSLVGLRPE
RYFWTGLSDVQNKGTFQWTIAEGVQFTHWNTDMPGRKAGCVAMRTGVAGGLWDVLRCEEKNKFVCKHWAE
GVTRPPEPTTTPEPKCPEDWGTSTKTSLCFKLFAKGKHEKKTWFESRDFCRALGGDLASINNKEEQQAIW
RLVTASGSYHELFWLGLTYSSPSEGFTWSDGSPVSYENWAYGEPNNYQNVEYCGELKSDAGMSWNDINCE
HLNNWICQIRKGQTPKPEPTPAPQDNPPVTEDGWVIYKDYQYYFSKEKETMDKAREFCKKNFGDLVSIQS
ESEKKFLWKYVNKNDAQPAYFIGLLISLDKKFIWMDGSKVDYVAWAAGEPNFANDDENCVTMYTHSGFWN
DINCGYPNAFICQRHNSSINATVTPTIPSAPGGCKEGWNFYNDKCFKIFGFVEEERKNWQEARKACIGFG
GNLASIRNEKEQAFLTYHMKDSTFNAWTGLNDVNSEHTFLWTDGRGVHYTNWGKGYPGGRRSSLSYEDAD
CVVIIGGKSRDAGKWMDDICDNKRGYICQTLPDSSLPRSPTTIPTDGFIKYGESSYSLTKLKLQWHEAAD
YCKLHSSLIASILDPYSNAFAWMQMQALSEPVWIALNSNLTNNEYVWTDKWRVRYTNWAADEPRLKTACV
YMDLDGSWKTANCNESFYFFCKKSDETPATEPPQLPGRCPESEHTAWIPFHGHCYYIESSYTRNWGQASL
ECLRMGSSLVSIESAAESSFLSYRVEPLQSKTNFWIGLYRNVEGMWLWVNNNPVSFVNWNTGDPSGERND
CVALYASSGFWNNIHCSSYKGYICKRPKIVDAEPTHALVTTKADPRKMVTSKPSSNSAGVVVIVVLLILT
GAGFAAYFFYKKRRVHLPQEENFENTLYFNSASTPGASDTKDLIGNIEQNEHVVI
>gi|302565029|ref|NP_001180854.1| macrophage mannose receptor 1-like
protein 1 precursor [Macaca mulatta]
SEQ ID NO: 5
MRLPLLLVFASVIPGAVLLLDTRQFLIYNEDHKRCVEAVSPSAVQTAACNQDAESQKFRWVSESQIMSVA
FKLCLGVPSKTDWVAITLYACDSKSEFQKWECKNDTLLGIKGEDLFFNYGNRQEKNVMLYKGSGLWSRWK
IYGTTDNLCSRGYEAMYTLLGNANGATCAFPFKFENKWYADCTSAGRSDGWLWCGTTTDYDTDKLFGYCP
LKFEGSESLWNKDPLTSISYQINSKSALMWHQARKSCQQQNAELLSITEIHEQTYLTGLTSSLTSGLWIG
LNSLSFNSGWQWSDRSPFRYLNWLPGSPSAEPGKSCVSLNPGKNAKWENLECVQKLGYICKKGNTTLNSF
VIPSESDVPTHCPSQWWPYAGHCYKIHKDEKKIQRDALTACRKEGGDLASIHTIEEFDFIISQLGYEPND
ELWIGLNDIKIQMYFEWSDGTPVTFTKWLRGEPSHENNRQEDCVVMKGKDGYWADRGCEWPLGYICKMKS
RSQGPEIVEVEEGCKKGWKKHHFYCYMIGHMLSTFAEANQTCNNENAYLTTIEDRYEQAFLTSFIGLRPE
KYFWTGLSDIQTKGTFQWTIEEEVRFTHWNSDMPGRKPGCVAMRTGIAGGLWDVLKCDEKAKFVCKHWAE
GVTHPPKPTTTPEPKCPEDWGASSRTSLCFKLYAKGKHEKKTWFESRDFCRALGGDLASINNKEEQQTIW
RLITASGSYHELFWLGLTYGSPSEGFTWSDGSPVSYENWAYGEPNNYQNVEYCGELKGDPSMSWNDINCE
HLNNWICQIHKGQTPKPEPTPAPQDNPPVTEDGWVIYKDYQYYFSKEKETMDNARAFCKRNFGDLVSIQS
ESEKKFLWKYVNRNDAQSAYFIGLLISLDKKFAWMDGSKVDYVSWATGEPNFANEDENCVTMYSNSGFWN
DINCGYPNAFICQRHNSSINATTVMPTKPSVPSGCKEGWNFYNNKCFKIFGFVEEERKTWQEARKACIGF
GGNLVSIQNEKEQAFLTYHMKDSTFSAWTGLNDVNSEHTFLWTDGRGVHYTNWGKGYPGGRRSSLSYEDA
DCVVIIGGASNEAGKWMDDTCDGKRGYICQTRSDPSLTNPPATIQTDGFIKYGKSSYSLMRQKFQWHEAE
IYCKLHNSLIASILDPYSNAFAWLQMETSNERVWIALNSNLTDSQYTWTDKWRVRYTNWAADEPKLKSAC
AYLDLDGYWKTAYCNESFYFLCKRSDEIPATEPPQLPGRCPESDHTAWIPFHGHCYYIESSYTRNWGQAS
LECLRMGSSLVSIESAAESSFLSYRVEPLKSKTNFWIGLFRNVEGMWLWINNSPVSFVNWNTGDPSGERN
DCVALHASSGFWSNIHCSSYKGYICKRPKIIDAKPTHELLTTKADTRKMDPSKPSSSVAGVVIIVILLIL
TGAGLAAYFFYKKRRVHLPQEGAFENTLYFNSQSSPGTSDMKDLVGNIEQNEHAVI

### Example 2

### Identification of the Mannose Receptor (MR/CD206) as a Soluble Protein in Human Serum

Materials and Methods

Western Blotting

Serum (diluted 1:10) and cell lysate controls (human monocyte derived macrophages 7.8 μg protein/μl and T24 human bladder carcinoma cell line 6.7 μg/μl) was analyzed by Sodium dodecyl sulfate-polyacrylamide gel electrophoresis (4-12% Novex® NuPAGE® SDS-page Gel System). Proteins were blotted onto PDVF membranes, blocked for 1 h and incubated o/n at 4° C. with mouse anti MR (Acris Antibodies, Clone 7-450, catalogue number AM05589PU-S) based on the protocol provided by Novex®. The membrane was subsequently incubated for 1 h at RT with goat anti-mouse-HRP (DAKO P0447, diluted 1:4000) and developed using enhanced chemiluminescence.

Affinity Purification of sMR from Human Plasma

MR was then purified from a pool of human EDTA-stabilized plasma by affinity chromatography. For mannose affinity chromatography, plasma was diluted 1:1 with 60 mM Tris pH 7.7, 100 mM NaCl, 20 mM Calcium (Buffer A), centrifuged at 10,000 rpm at RT for 30 min, and the supernatant filtered twice (Millipore Membrane filters 3.0 μM cat no. SSWP04700 and 0.8 μM cat no. AAWP04700) before loading on to a 5 mL Mannan-agarose column (Sigma-Aldrich cat no. MFCD00213010). The column had been initially washed in 60 mM Tris pH 7.7, 100 mM NaCl and 60 mM Tris pH 7.7, 100 mM NaCl, 20 mM Calcium, equilibrated with 60 mM Tris pH 7.7, 100 mM NaCl, 200 mM Mannose, 20 mM EDTA, and hence pre-equilibrated with sixty mL 60 mM Tris pH 7.7, 100 mM NaCl, 20 mM Calcium. After loading of the plasma, the column was washed with fifty mL 60 mM Tris pH 7.7, 100 mM NaCl, 20 mM Calcium, and hence, the bound protein was eluted with 60 mM Tris pH 7.7, 100 mM NaCl, 20 mM EDTA and collected in 1 mL fractions. Fractions with the highest concentrations of MR (as determined by ELISA) were pooled, dialyzed against 5 mM Tris pH 7.7, 10 mM NaCl for 24 h at 4° C., and concentrated on Amicon centrifugal filter device (MWCO 50,000, 4,000 rpm, Millepore). For antibody affinity chromatography 1 mg of anti-MR antibody was conjugated on a 1 ml HiTrap NHS-activated column (Product no: 17-0716-01, GE Healthcare, Brøndby, Denmark) according to manufacturer's instructions. Briefly, 1 mg of anti-MR antibody (catalog No: AM05589PU-N, Acris Antibodies GmbH, Herford, Germany) was dialyzed against 0.2 M NaHCO3, 0.5 M NaCl, pH 8.3 and concentrated to 1 mg/ml. The antibody coupling was performed in this buffer for 1 h at 25° C. Deactivation of excess active groups and washing out of uncoupled ligand was done by alternating washes with 2×6 ml 0.5 M ethanolamine, 0.5 M NaCl, pH 8.3 and 0.1 M acetate, 0.5 M NaCl, pH 4 and finally equilibrating the column by washing with 10 ml PBS. Plasma was prepared by centrifugation and sterile filtration. Purifications were made on the antibody affinity column with a flow rate of approximately 1 ml/min. 2 ml of plasma was loaded on the column. The column was washed with 5 ml PBS and eluted in 1 ml fractions with 100 mM Citrate pH 3 into tubes containing 100 μl 1 M Tris pH 8. The purification was repeated with additionally 2×2 ml plasma. Fractions containing MR (as determined by ELISA) were pooled and concentrated on Amicon centrifugal filter device (MWCO 50,000, 4,000 rpm, Millepore).

The purified MR was subjected to 4-12% SDS-PAGE and the gel stained by Coomassie Brilliant Blue. Bands of approximately 170 kDa were cut from the gel and subjected to protein identification by mass spectrometry MALDI MS/MS at Alphalyse A/S (Odense, Denmark).

Briefly, reduced and alkylated protein samples were digested with trypsin and peptides purified on a ZipTip micropurification column before they were analyzed on a Bruker Autoflex Speed MALDI TOF/TOF instrument. Spectra were combined and used for protein database searching using Mascot software (MS/MS ion search, 1 miscleavage, Peptide Tolerance: 60 ppm, Mascot version 2.2.03). Furthermore, data was searched against NCBI and NRDB protein databases and also searched against specific Macrophage Mannose Receptor 1 precursor sequence.

Enzyme Linked Immuno-Sorbent Assay (ELISA) for sMR

Polyclonal Anti-human MMR Antibody (0.2 mg/ml R&D systems, cataloguenumber AF2534 diluted 1:225 in 20 mM Carbonat-bicarbonatbuffer pH 9.6 to 0.7 mg/ml) 100 μl was coated onto microtitre wells (Nunc Maxisorp) and incubated at 4° C. for >17 h. The wells were washed three times in PBS, and subsequently 100 μl of sample (diluted 1:50 in PBS-albumin (10 mM, 0.5 M NaCl, 0.1% (V/V) Tween20, 0.2% (W/V) bovine serum albumin (Sigma no. A-4503), pH 7.2) was added and incubated for 1 h. The wells were washed, and 100 μl of inhouse biotinylated monoclonal anti MR (Acris Antibodies, Clone 7-450, catalogue number AM05589PU-S) diluted to 0.1 mg/l in PBS/albumin was added and incubated for one h. After washing, 100 μl of avidin-lysosym mixture (12 ml POD 10/400 ph 7.4+120 μl Lysosym (Sigma Cat. Nr. 6876 dilution 20 mg/l)+12μ Avidin (Dako Cat. Nr. P 0364)) was added and incubated for one h. The wells were washed and 100 μl TMB One (KEM-EN-TEC, Cat. Nr. 4380 A) was added. After incubating for 30 min 50 μl 1 M phosphorous acid was added and the plates were read at 450/620 nm in a microtitre plate reader (Thermo, Multiscan Acsent)

Validation of ELISA

To study linearity, twofold dilutions (1:5-1:10,240) of serum from a patient with high sMR was made and samples analyzed in quadruplicates. Estimates of within run imprecision was obtained by analyzing two samples 40 and 34 times respectively in the same run. Total variation was determined by control samples included in each run (n=32). No reference method or reference material was accessible and instead, recombinant Human MR (R&D Systems Catalog Number: 2534MR) was used for calibration. Calibrators with concentrations ranging from 1 to 256 μg/l were included on each plate. The level of sMR was also determined in serum based reference standards obtained from the Danish Institute for External Quality Assurance for Laboratories in Health Care reference sera (DEKS) (REF 1). Recovery was estimated by adding 100 μl of 400 μg/l recombinant MR diluted in PBS to 10 patient samples (diluted 1:10 in PBS). Furthermore paired serum, EDTA-plasma, and heparin-plasma (Terumo Cooporation, Japan) was collected from 30 patients and sCD163 determined in duplicates. The limit of detection (LOD) was determined by the mean of the zero standard plus 5 times the SD of the zero standard. The intra-assay variation of a serum sample diluted to 1.31 μg/L and measured in quadruplicate was 11.2% CV with a 4.2% bias.

To estimate the stability of sMR, 3 pools of fresh plasma were prepared, and aliquots of 50 μl were taken from each pool and kept at RT, +6° C., −20° C. and −80° C. Samples were analyzed during a period of 270 days and to reduce influence of day-to-day variation, 4 control samples included in each run were used to calibrate the results. Seven aliquots were also taken from each of the three pools of plasma and frozen (−80° C.) and thawed (+20° C.) one to seven times, frozen at −80° C. and subsequently analyzed in the same run.

Blood Samples

Serum samples from 240 healthy Scandinavians were obtained from the NOBIDA biobank, established as part of the Nordic Reference Interval Project (NORIP) (REF2). Values of common biochemical analytes was obtainable on 219 of the samples from the NORIP project and used for studies of covariation with sMR. These values were measured by the participating laboratories and harmonized from measured reference samples. All measurements were performed on thawed serum. Data on age, sex, height, weight, alcohol consumption (0, 1−21, >21 units/wk), medication, and physical activity (strenuous exercise within last week) was also obtained from the NORIP project.

EDTA-stabilized surplus plasma samples from 218 patients at Aarhus University Hospital were collected after routine testing at the Department of Clinical Biochemistry. These samples were collected over a period of two months and frozen at −80° until they were analyzed. Clinical data and routine lab results were obtained from the medical files of the patients. Soluble CD163 was measured essentially as previously described (REF3). Furthermore, 51 patients with acute alcoholic hepatitis were prospectively enrolled at the department of Gastroenterology and Hepatology, Aarhus Denmark. Peripheral venous blood samples were collected upon diagnosis and before initiation of treatment. After centrifugation, plasma was stored at −80° C. for up to 20 months until analysis. Fifty samples were available for sMR measurements.

Ethics

The local Ethical Committee of Central Denmark Region approved the study of patients with hepatitis (M-20080203) and informed consent was obtained from all participants. According to Danish law, the collection of surplus patient samples did not need to be approved by the Regional Scientific Ethical Committee. The reviewing of patient records was approved the Danish National Board of Health (Journal number 7-604-04-2/300) and by the Danish Data Protection agency (Journal number 2007-58-0010).

Statistical Analyses

Probability plots showed that all data were normally distributed either as raw data or after log transformation. The following data was log transformed in the reference group: BMI, Cholesterol, HDL Cholesterol, LDL Cholesterol, Triglycerides, Glucose, Uric acid, ALT, AST, GGT, ALP, Bilirubin. The following data was log transformed in the patient group: ALT, ALP, LDH, CRP, sCD163, Albumin, Hemoglobin, Leukocytes, Lymfocytes, Monocytes, Neutrofils, Imature granulocytes. Differences within unpaired data were evaluated with a two-sided t-test and paired data were evaluated with a paired student's t-test. The 95% reference interval was determined by calculating mean+/−1,96*σ. P-values less than 0.05 were considered statistically significant. Statistical analysis was performed using GraphPad Prism 4 and Analyse-it for Microsoft Excel.

Results

The Mannose Receptor is Present as a Soluble Protein in Human Plasma

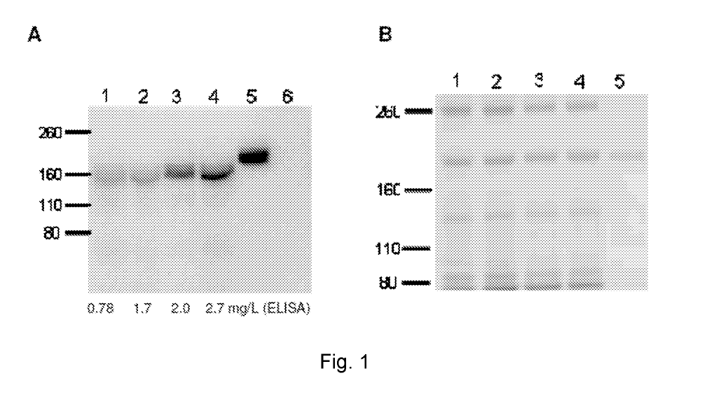

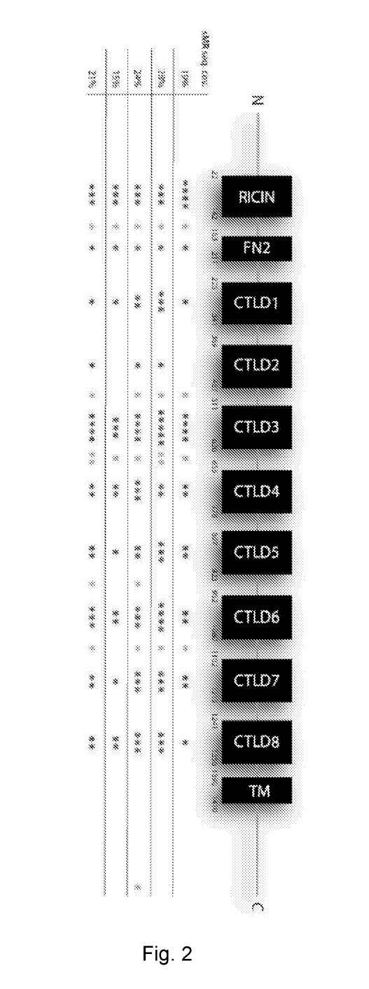

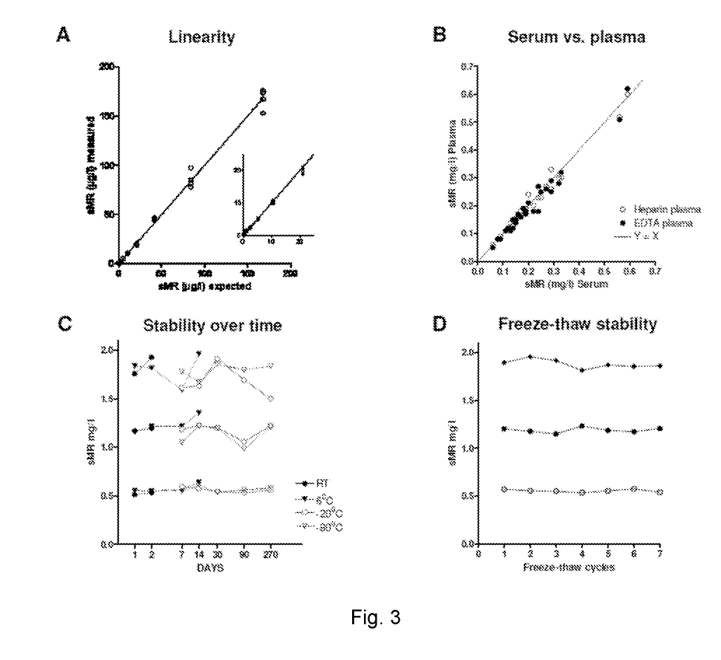

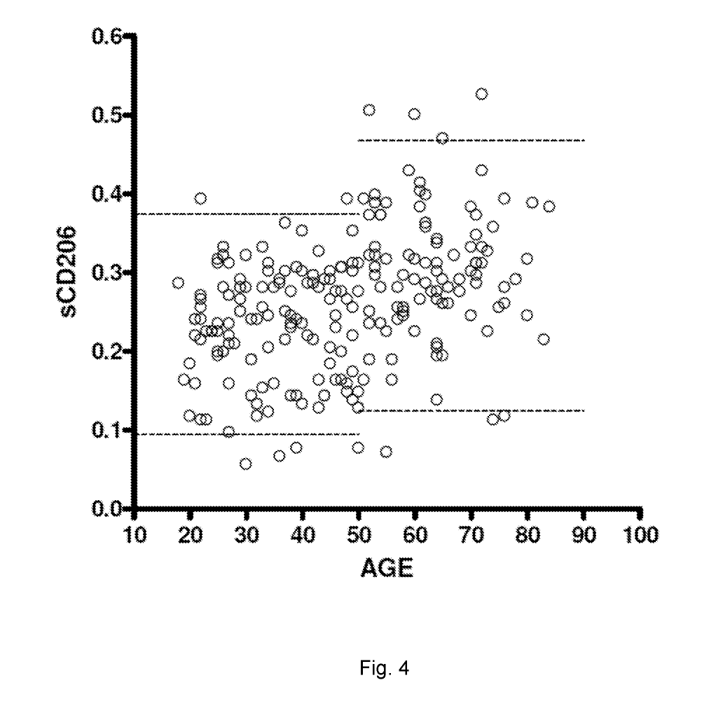

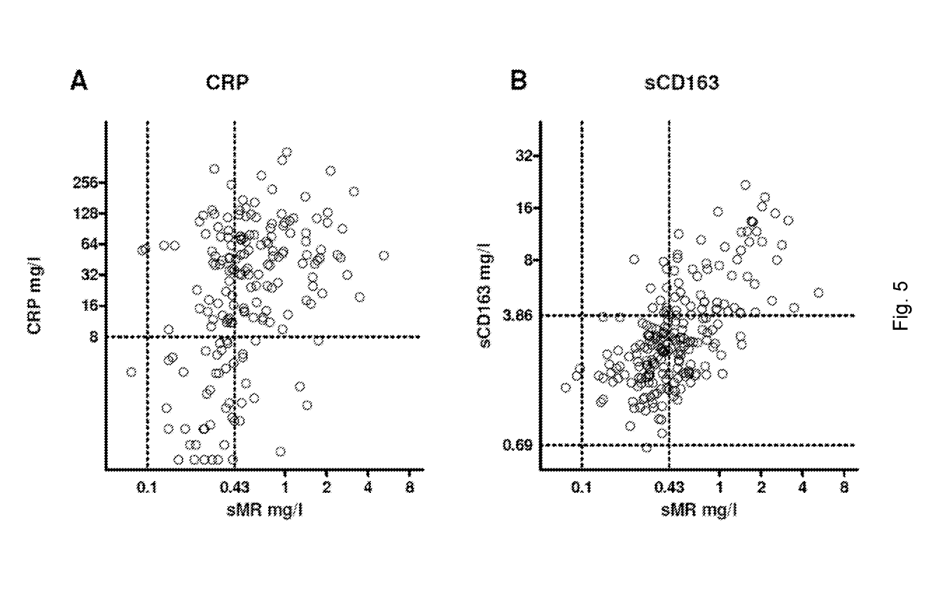

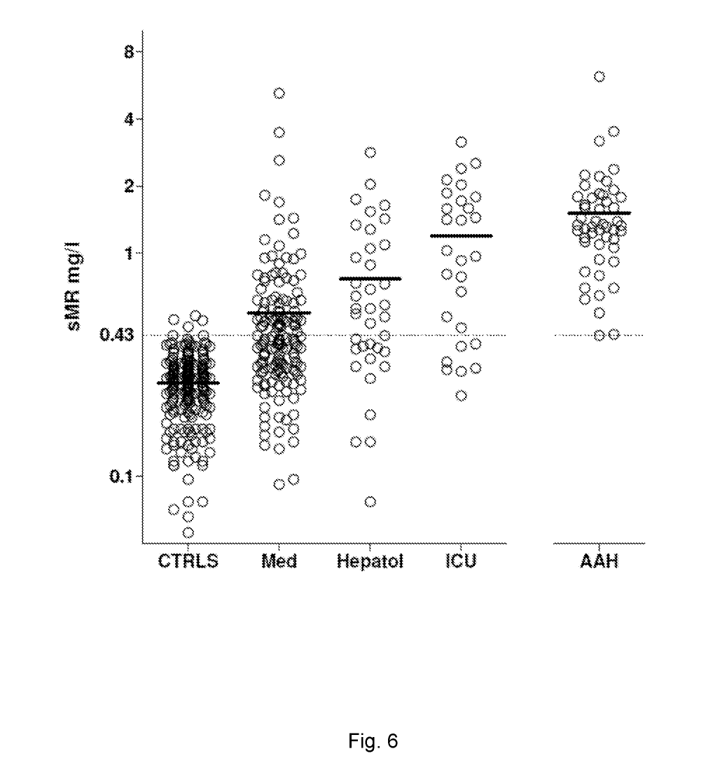

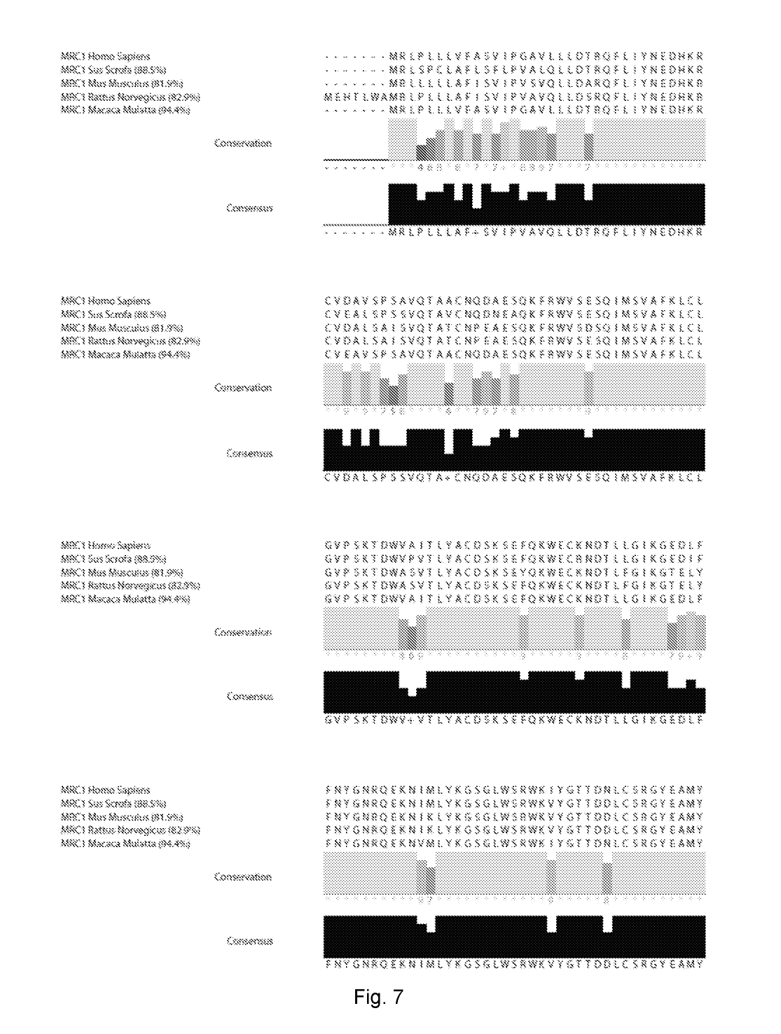

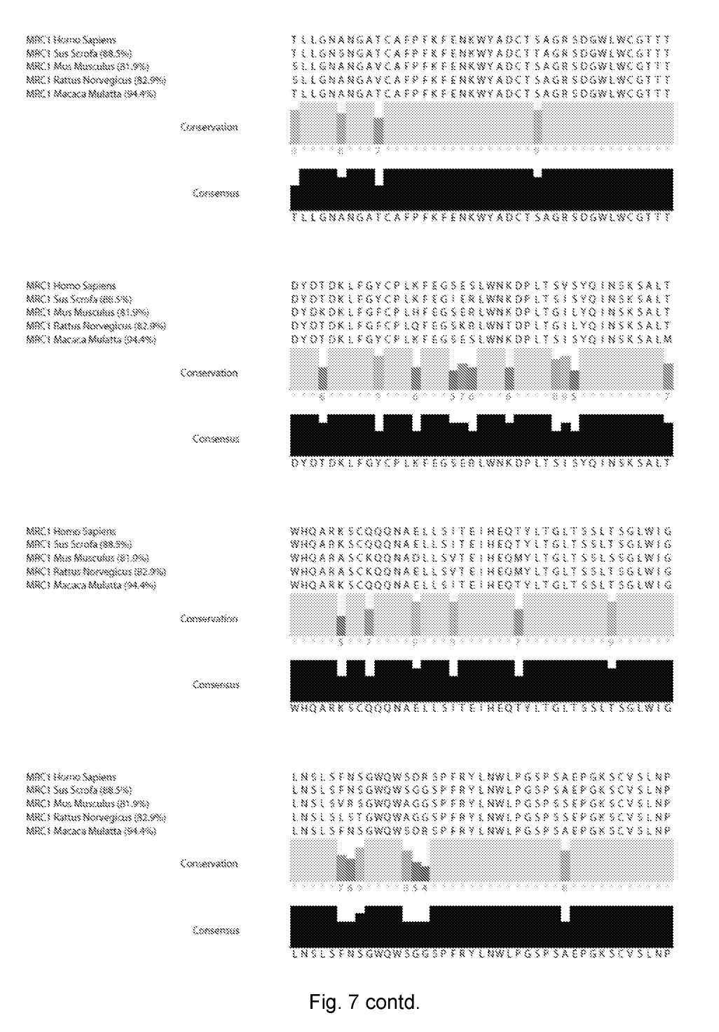

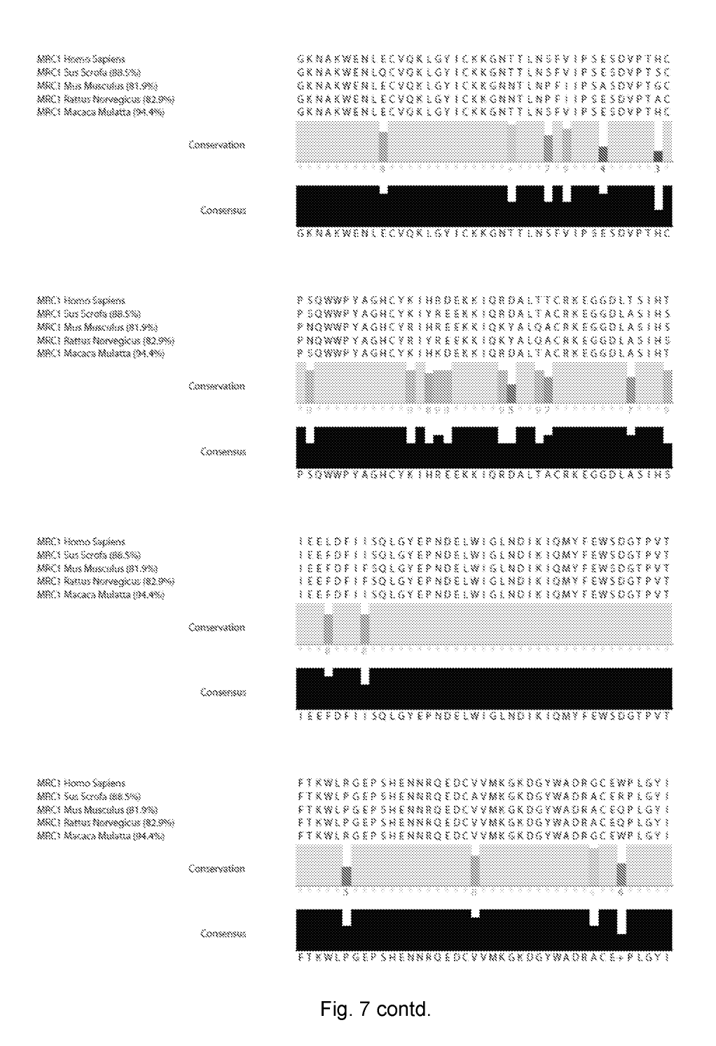

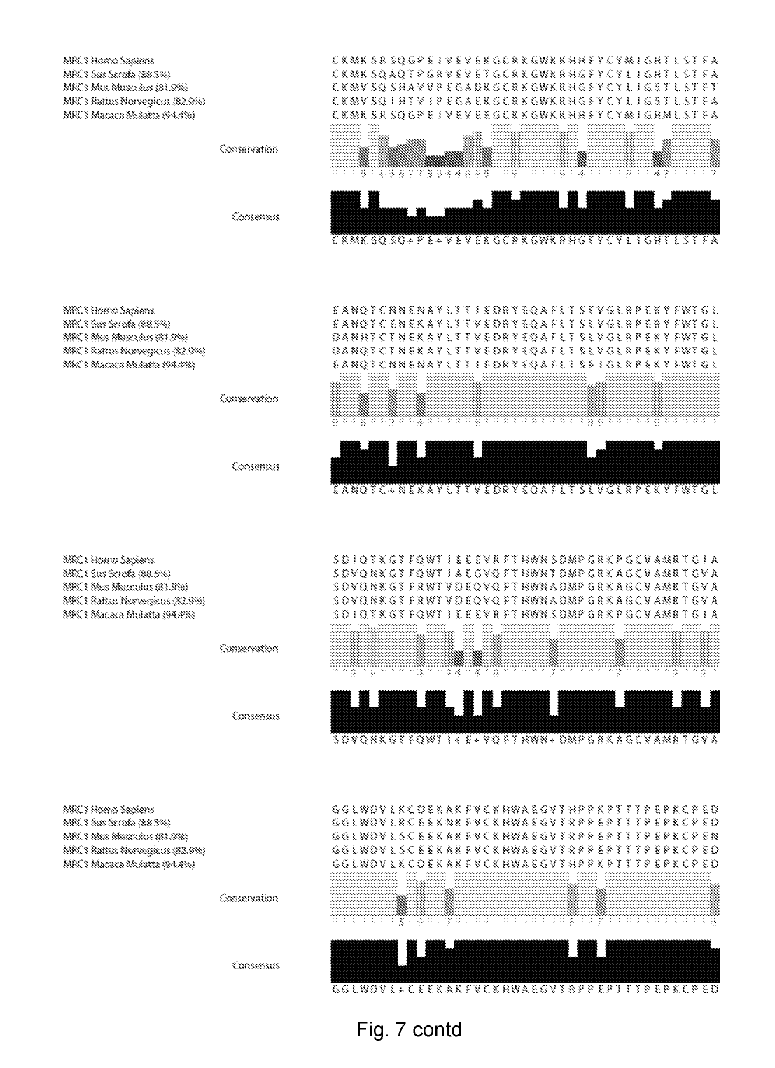

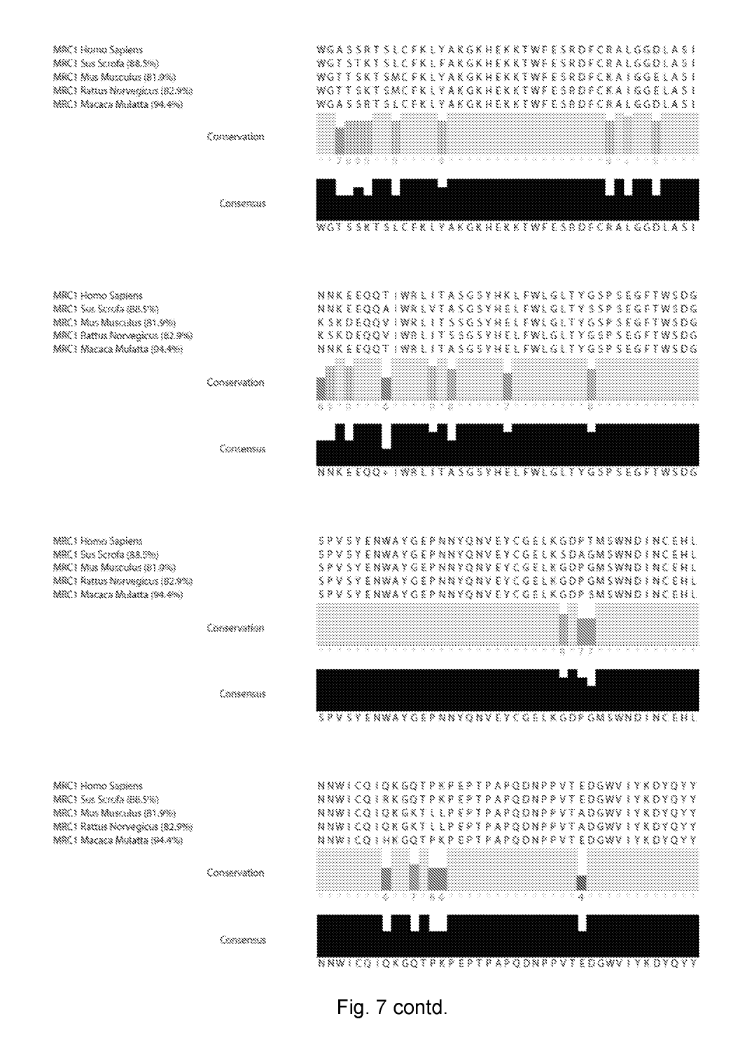

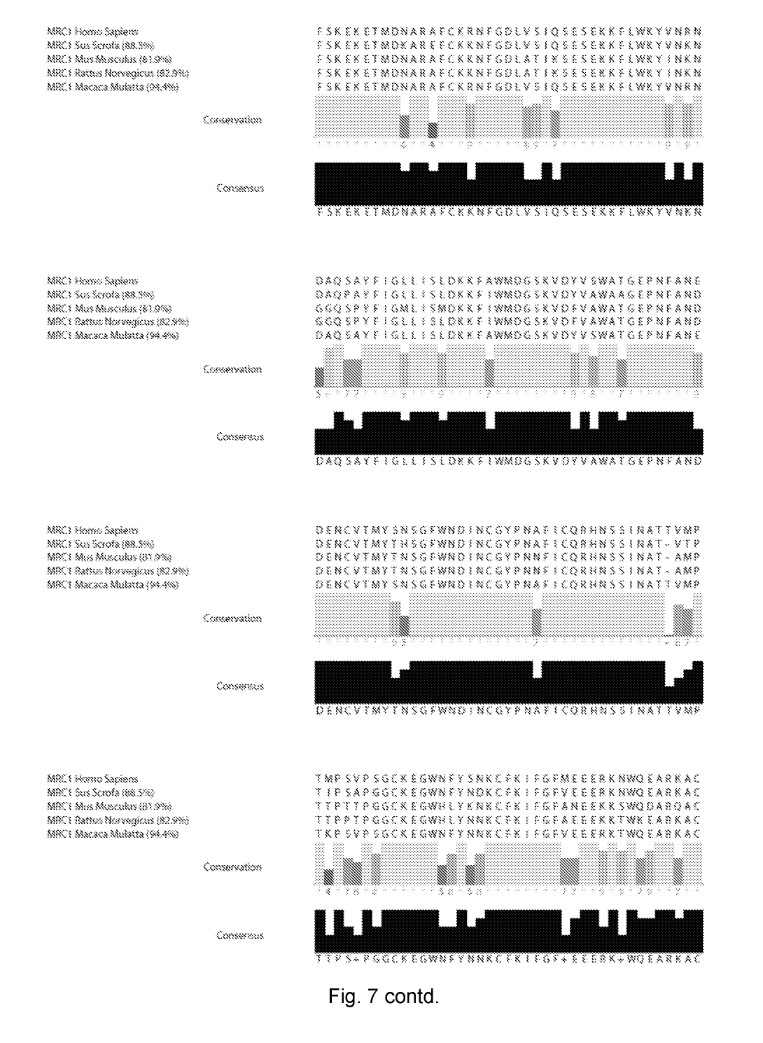

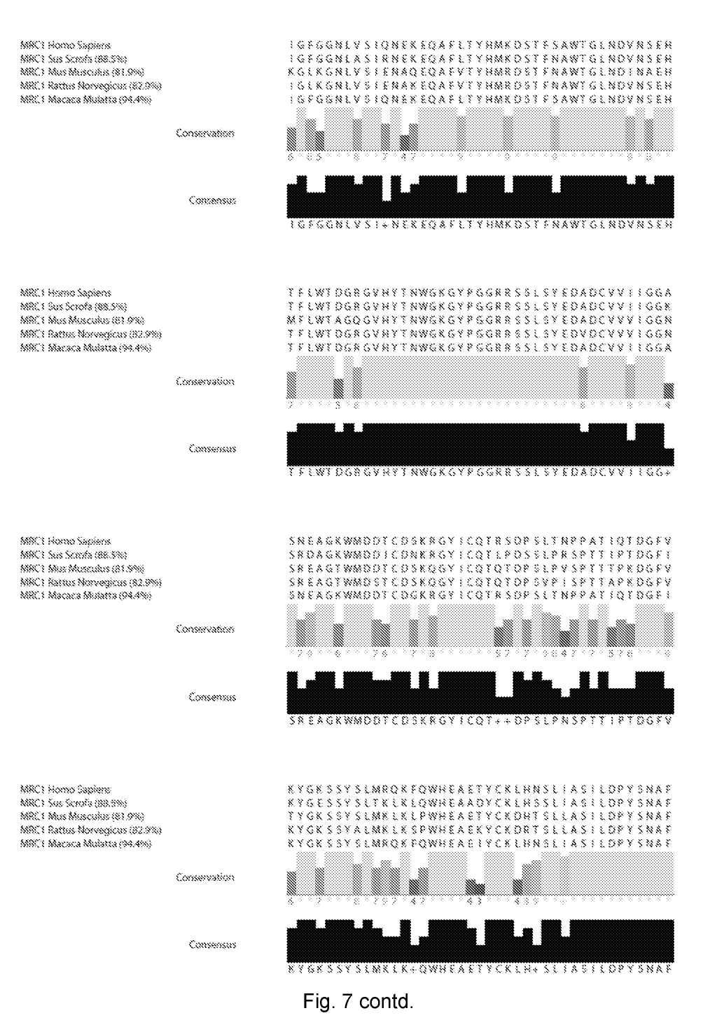

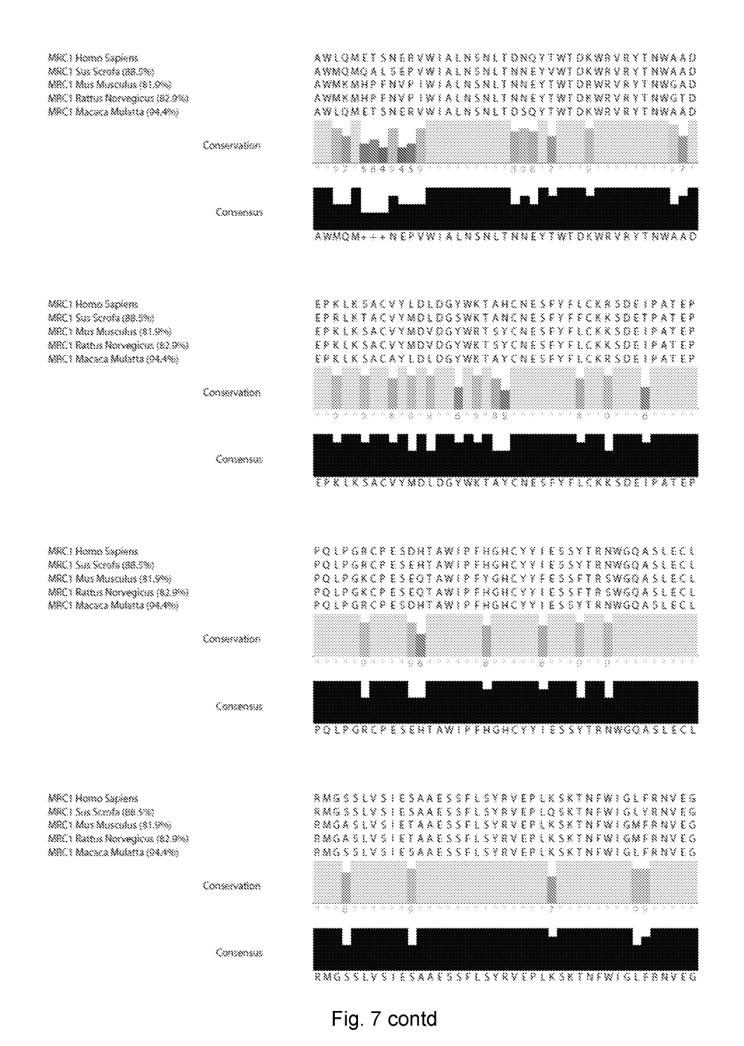

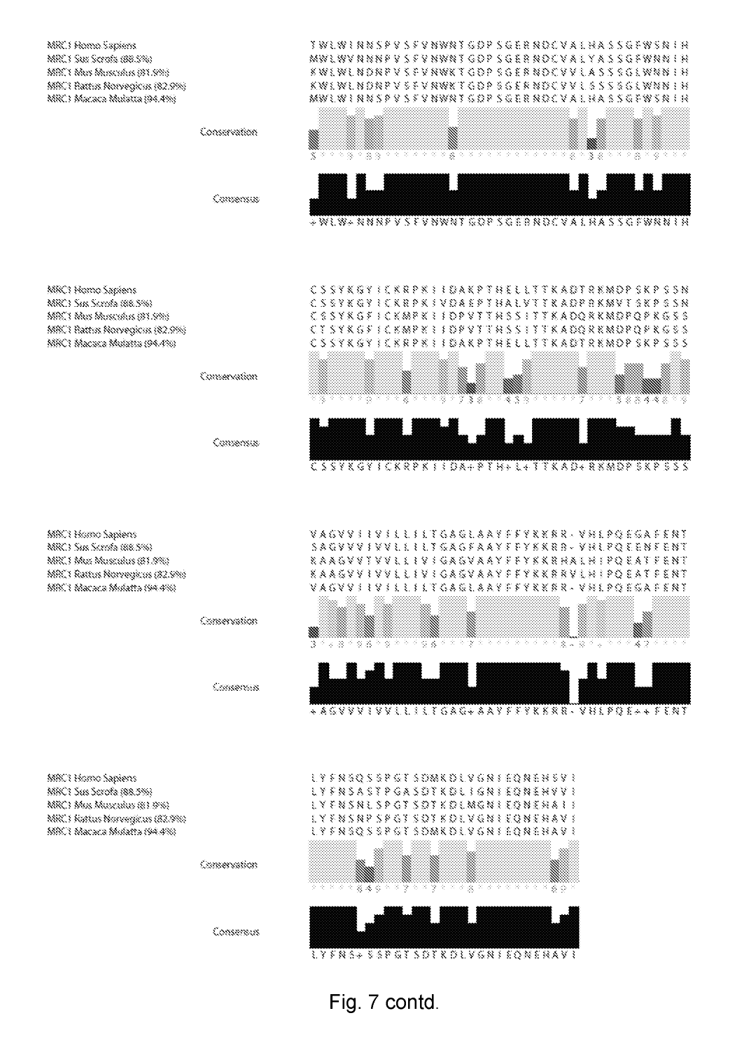

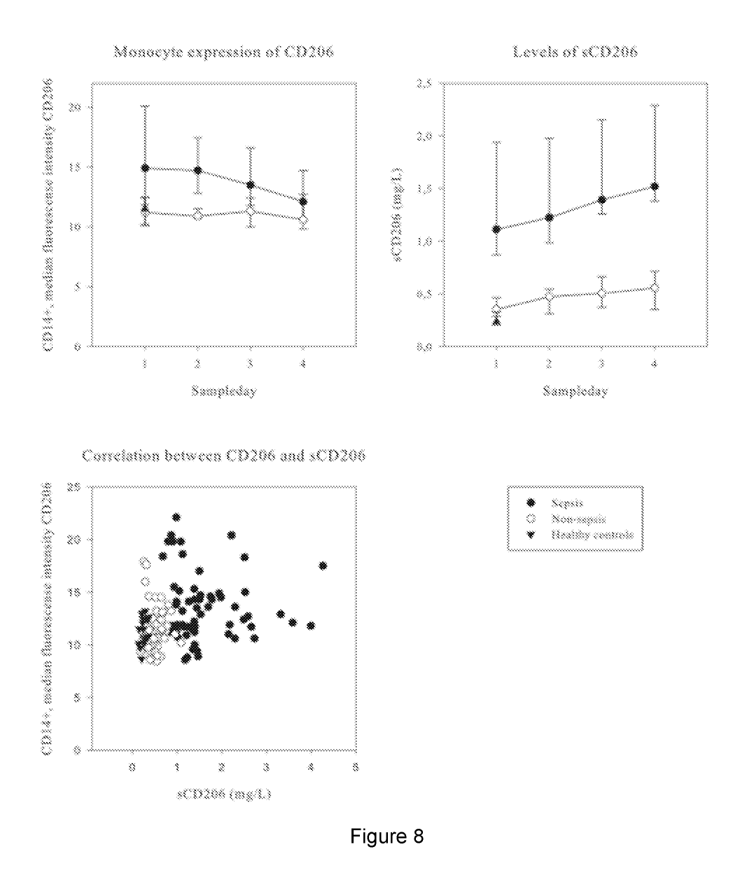

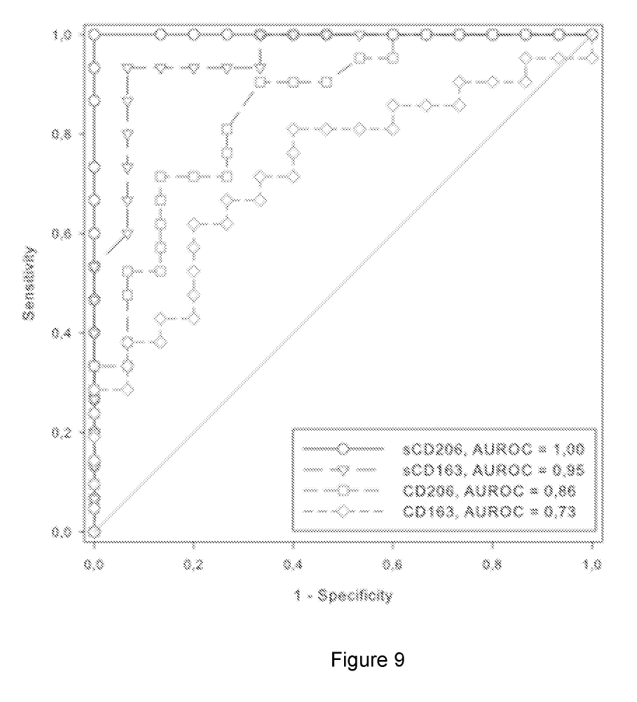

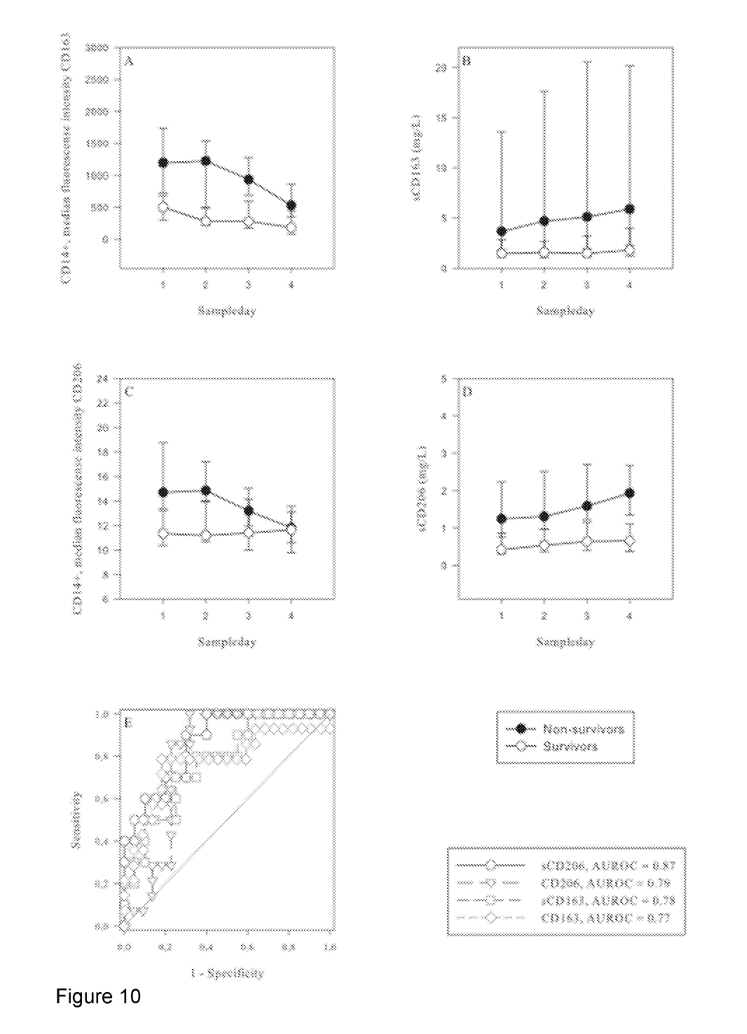

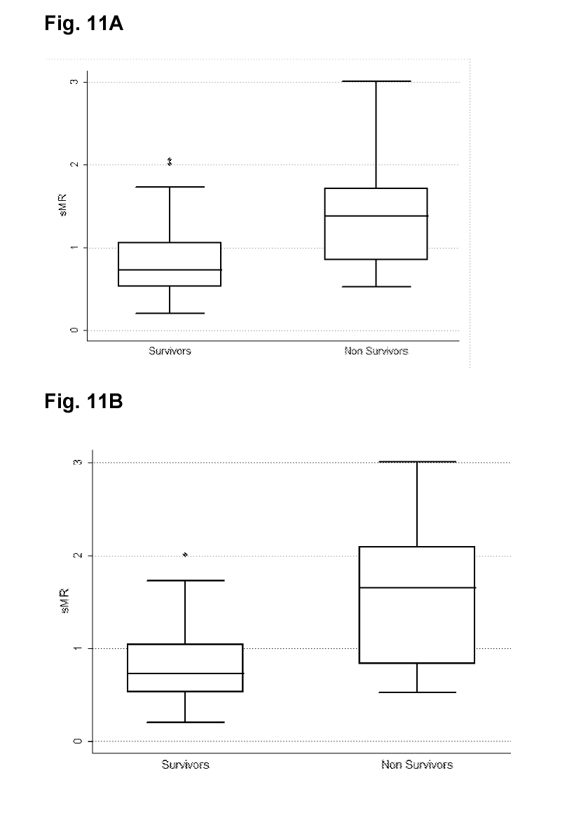

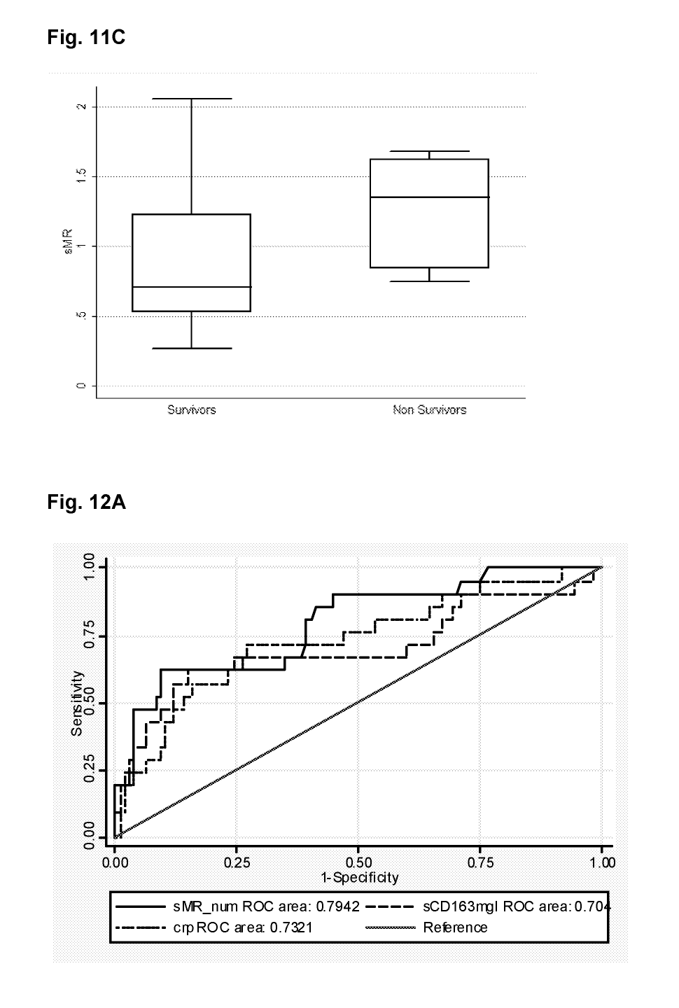

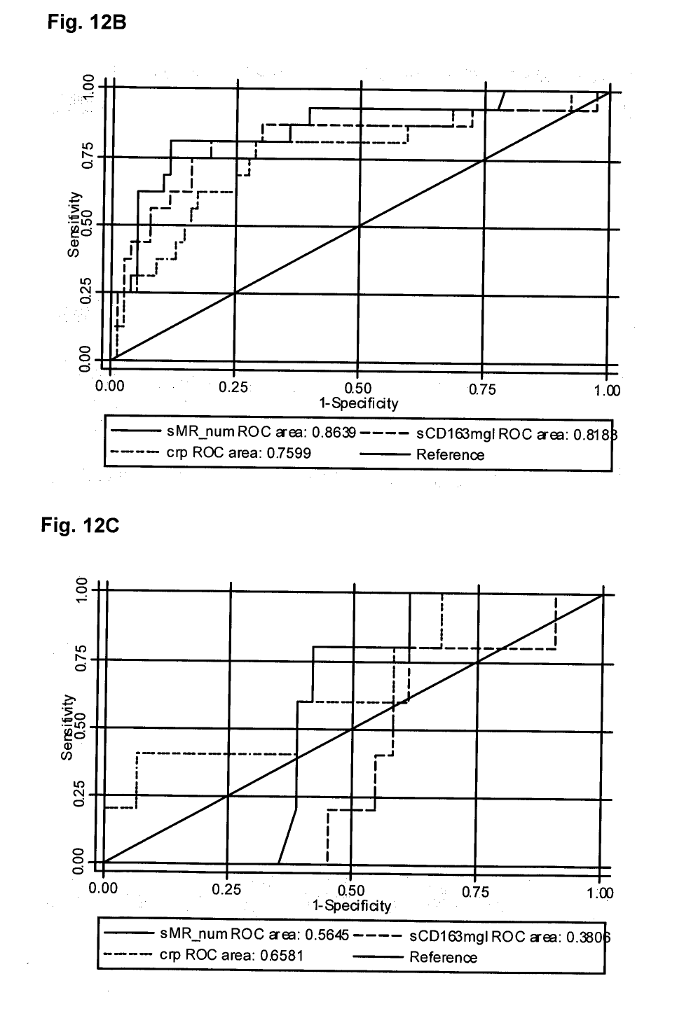

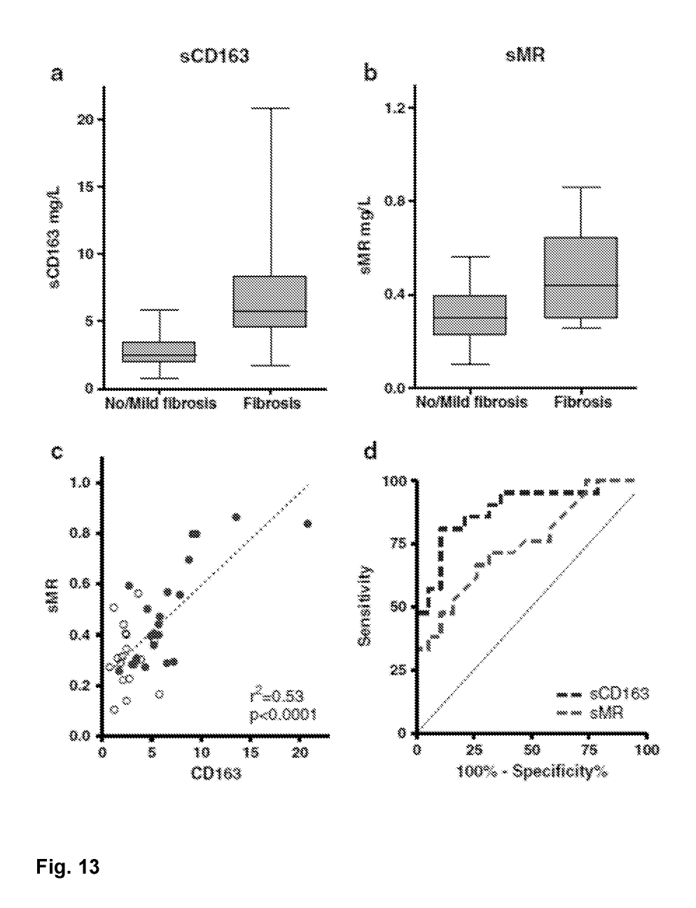

We examined human sera and cell-extracts by SDS-gel electrophoresis followed by immunoblotting and identified a soluble form of MR in serum (FIG. 1A). A single band with a molecular weight close to that of the full-length membrane form of MR was detected in all sera (lanes 1-4). The mobility was slightly faster than the MR reactivity of a human macrophage lysate (lane 5). No reactivity was seen to human bladder cancer cells (lane 6). Next we purified sMR by both mannose affinity chromatography, and immune-affinity chromatography and the eluted proteins were analyzed by SDS-PAGE (FIG. 1B). The protein bands corresponding to approximately 170 kDa (lanes 2-6) were cut and subjected to MALDI MS/MS. Peptide mapping and sequencing analysis identified macrophage mannose receptor (MRC1) in all 5 preparations. Sequence coverage ranged from 15 to 28%, and multiple peptide sequences were identified in the N-terminus cysteine rich ricin b-type lectin domain (RICIN), the fibronectin type-II domain (FN2) and in all the C-type lectin-like domains 1-8 (CTLD) including the functional Ca²⁺ dependent mannose-binding CTLD 4 domain in the extracellular part of the protein (FIG. 2). These peptides were spanning aminoacids 24-1301 in the extracellular part of the protein, 88 aminoacids from the transmembrane domain, indicating a putative cleavage site between CTLD domain 8 and the transmembrane domain (amino acid 1390-1410). Interestingly, in one of the bands an intracellular C-terminal sequence (amino acid 1443-1456) was identified (FIG. 2).

Establishment and Validation of an ELISA for Measurement of sMR in Human Serum

We explored commercially available antibodies against sMR and established and optimized an ELISA using a polyclonal coating-antibody and a monoclonal biotinylated secondary antibody. The assay was linear in serum dilutions ranging from 1:20-1:2560 (slope 1.003 (95% CI 0.978-1.027), intercept 0.171 (−1.35-1.69), R²=0.99 covering a concentration range of 1.3-168 μg/l (FIG. 3A). Within run imprecision was 8.9% CV at a concentration of 0.35 mg/l (n=40) and 3.0% CV at a concentration of 2.35 mg/l (n=34). Total imprecision was between 5.9 and 11% CV estimated by analyzing control samples in 32 runs. A recombinant human MR protein was used to calibrate the assay and used to establish the concentration in 3 normal control sera from the Danish Institute for External Quality Assurance for Laboratories in Health Care (Control-X 0.27 mg/L (+/−0.035), control-Low 0.16 mg/L (+/−0.016) control-High 0.32 mg/L (+/−0.036). Mean recovery of recombinant MR added to patient samples was 114% (n=10, range 73-150). There was an excellent correlation between plasma and serum concentrations in pared samples from 30 patients (R²=0.97-0.98, FIG. 3B), however concentrations in EDTA-plasma were 3-4% lower than in heparin-plasma and serum (p=0.011 and 0.013 respectively). The limit of detection (LOD) was 0.5 μg/L and limit of quantitation (LOQ) below 1.3 μg/l which is 46 times lower than the lowest concentration detected in more than 400 serum samples (see below).

In pools of EDTA-plasma sMR was stable for more than two days at RT, for 2 weeks at 6° C., and for at least 9 months at −20° C. and −80° C. respectively (FIG. 3C). The concentration of sMR was found to be stable through at least six rounds of freezing and thawing (FIG. 3D).

Serum Concentration of sMR in Healthy Individuals

We measured sMR in sera from 219 healthy Scandinavian individuals. Two samples with very high concentrations (2.6 and 4.8 mg/l respectively) were considered to be outliers and excluded from further analyses. The remaining 217 samples had levels between 0.06-0.53 mg/l and showed a Gaussian distribution (evaluated by histogram and QQ-plot). The mean concentration (n=217) was 0.28 mg/l (SD 0.085) and a parametric 95% reference interval was established to 0.10 mg/l (90% CI 0.08-0.11) to 0.43 mg/l (90% CI 0.41-0.45). There was a weak but significant, positive correlation between sMR and age (R²=0.13, p<0.0001). When stratifying for age under/over 50 years, the reference intervals were significantly different, <50 years: 0.09-0.37 mg/l 50 years: 0.12-0.46 mg/l (FIG. 4). There was no significant difference between the sMR concentrations in men and women (p=0.31), and the sMR level was not influenced by reported oestrogen treatment (p=0.14), alcohol intake (p=0.37), or physical activity (p=0.45). sMR correlated weakly with metabolic markers and some liver markers (table 1) but not with markers of kidney function, iron-status, bone-metabolism, or thyroid function.

Serum Concentration of sMR in Hospitalized Patients

In order to get a first impression as to the concentrations of sMR in relation to pathological conditions we examined serum from 218 unselected hospitalized patients. Of these, 110 (50.5%) had concentrations above the upper reference range (>0.43 mg/l). There was a significant correlation between sMR and biochemical markers of liver disease and inflammation (Table 2). The correlation to the general inflammatory marker CRP was moderate (R²=0.11, P<0.0001), and it seemed that patients with increased sMR constituted a fraction of patients with increased CRP (FIG. 5A). sMR correlated strongly to a specific macrophage activation marker sCD163 (R²=0.53, p<0.0001), however a higher fraction of sMR values were above upper reference range (50.5%, n=110) than for sCD163 (31%, n=68) (FIG. 5B). Reviewing patient files revealed that very high levels were typically seen in critically ill patients with sepsis and/or severe liver disease, and accordingly, the highest concentrations were measured in patients from the ICU (median 1.00 mg/l, 10 of 20 highest values) and department of hepatology and gastroenterology (median 0.57 mg/l, 5 of 20 highest values) (FIG. 6). We therefore measured sMR concentrations in a well characterized population of patients (n=50) with acute alcoholic hepatitis, a severe liver disease with high mortality. The mean level in these patients was 1.52 mg/l (range 0.43-6.21 mg/l) (FIG. 6).

REF1. Danish Institute for External Quality Assurance for Laboratories in Health Care, DEKS. Accessed at http://www.deks.dk/index.html

Oct. 1, 2012.

REF2. Nordic Reference Interval Project. Accessed at http://pweb.furst.no/norip/

Oct. 1, 2012.

REF3. Møller H J, Hald K, Moestrup S K. Characterization of an enzyme-linked immunosorbent assay for soluble CD163. Scand J Clin Lab Invest 2002; 62:293-299.

TABLE 1
Correlation of sMR to selected biochemical parameters in healthy
individuals

| | | | |
|---|---|---|---|
| Parameter | R² | P | n |

Metabolic

| | | | |
|---|---|---|---|
| BMI | 0.06 | 0.002 | 214 |
| S-Cholesterol | 0.06 | 0.0002 | 214 |
| S-HDL cholesterol | −0.02 | NS | 209 |
| S-LDL cholesterol | 0.11 | 0.0016 | 90 |
| S-Triglycerides | 0.008 | 0.008 | 90 |
| S-Glucose | 0.12 | 0.003 | 69 |
| S-Uric acid | 0.003 | 0.01 | 208 |

Liver

| | | | |
|---|---|---|---|
| S-ALT | 0.005 | NS | 183 |
| S-AST | 0.05 | 0.0019 | 174 |
| S-GGT | 0.04 | 0.03 | 127 |
| S-ALP | 0.02 | NS | 72 |
| S-Bilirubin | 0.005 | NS | 215 |
| S-Albumin | −0.04 | 0.0026 | 213 |

S; Serum,
BMI; Body Mass Index,
HDL; High density lipoprotein,
LDL; Low density lipoprotein,
ALT; Alanine Aminotransferase,
AST; Aspartate Aminotransferase,
GGT; Gamma-glutamyltransferase,
ALP; Alkaline phosphatase.

TABLE 2
Correlation of sMR with biochemical markers in hospitalized patients

| | | | |
|---|---|---|---|
| Parameter | R² | P | n |

Liver

| | | | |
|---|---|---|---|
| P-ALT | 0.12 | 0.0009 | 84 |
| P-ALP | 0.29 | <0.0001 | 85 |
| P-Bilirubin | 0.11 | 0.004 | 75 |
| P-LDH | 0.21 | 0.0002 | 62 |
| P-PT | −0.18 | 0.0009 | 59 |

Inflammation

| | | | |
|---|---|---|---|
| P-CRP | 0.11 | <0.0001 | 174 |
| S-sCD163 | 0.53 | <0.0001 | 217 |
| P-Albumin | −0.41 | <0.0001 | 120 |
| B-Hemoglobin | −0.14 | <0.0001 | 194 |

Cells

| | | | |
|---|---|---|---|
| B-Leukocytes | 0.09 | <0.0001 | 209 |
| B-Lymphocytes | −0.05 | 0.003 | 174 |
| B-Monocytes | 0.07 | 0.0005 | 174 |
| B-Neutrophiles | 0.1 | <0.0001 | 174 |
| B-Immature gran | 0.17 | <0.0001 | 145 |
| B-Thrombocytes | 0 | NS | 157 |

Electrolytes

| | | | |
|---|---|---|---|
| P-Potassium | 0 | NS | 185 |
| P-Sodium | −0.02 | NS | 183 |
| P-Magnesium | 0.02 | NS | 28 |
| P-Calcium | −0.12 | 0.03 | 40 |
| P-Phosphate | 0.03 | NS | 32 |
| P-Creatinine | 0.01 | NS | 196 |

P; Plasma,
S; Serum,
B; Blood,
gran: granulocytes,
ALT; Alanine Aminotransferase,
AST; Aspartate Aminotransferase,
LDH; Lactate dehydrogenase,
PT; prothrombin time,
CRP; C-reactive protein.
Only selected parameters with N >25 are shown

### Example 3

### Identification of sCD206 as a Marker for Sepsis

Materials and Methods

Study design & patients

Prospective observational study carried out at one mixed Intensive Care Unit (ICU) and one neurosurgical ICU at Aarhus University Hospital and at one mixed ICU at Randers regional Hospital, Denmark. Informed consent was obtained from the subjects if possible, alternatively from the closest relative and the patients' general practitioner prior to the study. The study was approved by the local ethics committee. 30 ICU patients were included in the study between January 2010 and January 2012. 15 patients with severe sepsis or septic shock by Bones's definition [Ref 4] later modified by Levy and collegeaus [Ref 5] were included. The diagnosis of sepsis was applied when the patient as a response to a documented or clinical suspected infection fulfilled 2 or more of the following systemic inflammatory response syndrome (SIRS) criteria: 1) temperature above 38° C. or below 36° C., 2) heart rate above 90 beats per minute, 3) respiratory rate above 20 breaths per minute or PaCO₂ below 32 mmHg, and 4) white blood cell count above 12,000 cells/μl or below 4,000 cells/μl, or more than 10% immature forms. The term severe sepsis was used when sepsis was associated with organ dysfunction, hypoperfusion, or hypotension. Criteria for organ dysfunction was obtained from Marshall and colleagues [Ref 6]. Septic shock was defined as severe sepsis with hypotension (systolic blood pressure below 90 mmHg) despite adequate fluid resucitation. In addition 15 severely ill non-septic patients were included. Severely ill was defined as an Acute Physiology And Chronic Health Evaluation (APACHE II) score above 13 at ICU admission. 15 healthy volunteers matched on age and gender served as a control group.

Exclusion criteria were age below 18, pregnant or lactating, hematocrit below 0.30, immunemodulating therapy except for low dose steroid, chemotherapy or radiationtherapy within one year of inclusion, lifethreathning bleeding, inclusion in medical intervention research, brain-dead at time of inclusion, expected survival shorter than 4 days or expected ICU admission shorter than 4 days, and lack of informed consent or withdrawal of informed consent.

The sequential organ failure assessment (SOFA) score [Ref 7] was calculated daily to determine the extent of organ failure. The SOFA score is based on scores from six organ systems (cardiovascular, coagulation, hepatic, neurological, renal, and respiratory) and gives an estimate of organ dysfunction.

The acute physiology and chronic health evaluation II (APACHE II) score [Ref 8] was calculated on ICU admission. The APACHE II score is based on 12 routine physiologic measurements, age, and previous health status. The score gives an estimate of severity of disease and a predicted death rate.

Blood Sampling

Blood samples were drawn from an arterial cannula on day 1, 2, 3, and 4 of ICU admission. The samples were drawn between 8 am and 12 am to avoid circadian variation in the measured parameters. Routine blood samples were analysed immediately by the local biochemical department.

Results

Patient Characteristics

Important patient characteristics at baseline are shown in tables 3 and 4. All three groups were similar with regard to age, sex, alcohol consumption, blood sample time, and temperature.

The septic group had higher respiratory rate (RR) and heart rate (HR) than the non-septic group and control. The latter two did not differ with regard to RR and HR.

There was no difference between the Septic and non-septic group with regards to APACHE II score, SOFA score at ICU admission, and white blood cell (WBC) count. As expected these variables were found to be lower in the control group. Mean arterial pressure (MAP) was lower in the septic group compared to controls and the non-septic group, which again had lower MAP than controls.

Patients in the septic group smoked as much as patients in the non-septic group, but more than the healthy controls. There was no difference between non-septic patients and healthy controls.

The septic and non-septic groups were similar with regards to PaCO₂, type of ventilation, and need of dialysis. The septic group was more likely to receive inotropic agents, antibiotics, glucocorticoids, and to be on a euglycemic clamp, than the non-septic group

sCD206 in Septic, Severely Ill Non-Septic ICU Patients, and Healthy Controls

At ICU admission, levels of sCD206 were significantly higher in patients with severe sepsis and septic shock compared to severely ill non-septic patients and healthy controls (p<0.001 for all comparison). Levels of sCD206 were higher in the non-septic patients compared to controls (p=0.002). During the four-day observation period, levels of sCD206 were significantly higher in the septic patients compared with the severely ill non-septic patients (p<0.001 for both comparisons) (FIG. 8).

To examine if the monocyte expression of CD163 and levels of sCD163 and sMR could be used to discriminate between septic patients and non-septic patients, we performed AUROC curve analysis. At ICU admission, sMR had the highest AUROC (1; 95% CI 1 to 1), followed by sCD163 (0.95; 95% CI 0.88 to 1) and monocyte-bound CD163 expression (0.75; 95% CI 0.58 to 0.91) (FIG. 9 and Table 5). There was no difference between sMR and sCD163, and both markers performed better than monocyte-bound CD163. The AUROC for sMR was significantly higher than the AUROC for plasma CRP (0.87, 95% CI 0.76 to 0.99) (p=0.04). There was no difference in the AUROC between plasma CRP and CD163 or between plasma CRP and sCD163.

sMR at a cut-off value of 0.61 mg/ml were able to discriminate between septic and non-septic patients with 100% sensitivity and 100% specificity. sCD163 at a cut-off value of 1.74 mg/ml had 93% sensitivity and 93% specificity. CD163 at a MFI cut-off value of 646.5 had 67% sensitivity and 80% specificity. CRP at a cut-off value of 91.8 mg/l had 80% sensitivity and 86% specificity.

At ICU admission, sCD206 (p=0.001) was higher in in-hospital non-survivors than survivors (FIG. 10 D & Table 5). At ICU admission sCD206 had an AUROC=0.87 to predict mortality (95% CI 0.74 to 0.99) (FIG. 10 E and Table 5). Recalculation of AUROC on day 2, 3, and 4 did not improve these results (Table 5).
Ref 4: Bone R C, Balk R A, Cerra F B, Dellinger R P, Fein A M, Knaus W A, Schein R M, Sibbald W J, (1992) Definitions for sepsis and organ failure and guidelines for the use of innovative therapies in sepsis. The ACCP/SCCM Consensus Conference Committee. American College of Chest Physicians/Society of Critical Care Medicine. Chest 101: 1644-1655
Ref 5: Levy M M, Fink M P, Marshall J C, Abraham E, Angus D, Cook D, Cohen J, Opal S M, Vincent J L, Ramsay G, (2003) 2001 SCCM/ESICM/ACCP/ATS/SIS International Sepsis Definitions Conference. Intensive Care Med 29: 530-538
Ref 6: Marshall J C, Cook D J, Christou N V, Bernard G R, Sprung C L, Sibbald W J, (1995) Multiple organ dysfunction score: a reliable descriptor of a complex clinical outcome. Crit Care Med 23: 1638-1652
Ref 7: Vincent J L, Moreno R, Takala J, Willatts S, De Mendonca A, Bruining H, Reinhart C K, Suter P M, Thijs L G, (1996) The SOFA (Sepsis-related Organ Failure Assessment) score to describe organ dysfunction/failure. On behalf of the Working Group on Sepsis-Related Problems of the European Society of Intensive Care Medicine. Intensive Care Med 22: 707-710
Ref 8: Knaus W A, Draper E A, Wagner D P, Zimmerman J E, (1985) APACHE II: a severity of disease classification system. Crit Care Med 13: 818-829

TABLE 3
Patient characteristic at admission

| | Sepsis | Non-sepsis | Control |
|---|---|---|---|
| | n = 15 (21) | n = 15 | n = 15 |
| Age, median (IQR) | 66 (62-79) | 58 (47-68) | 61 (59-63) |
| Sex (males/females) | 8/7 | 7/8 | 6/9 |
| Temp (° C.), | 38.1 (36.9-38.5) | 37.5 (36.6-37.9) | — |
| median (IQR) | | | |
| HR, median (IQR) | 113 (102-129) | 71 (66-83) | 65 (58-71) |
| RR, median (IQR) | 22 (18-30) | 15 (14-21) | 16 (15-17) |
| PaCO₂ (kPa) | 6 (5.2-7.5) | 5.7 (5.3-5.9) | — |
| WBC (×10⁹/l) | 12.5 (8.4-21.5) | 12.1 (10.5-14.1) | 5.1 (4.5-6.6) |
| MAP (mmHg), | 76 (64-81) | 84 (82-99) | 104 (95-108) |
| median (IQR) | | | |
| APACHE II score, | 17 (15-22) | 18 (15-23) | 5 (5-6) |
| median (IQR) | | | |
| SOFA score, | 8 (7-12) | 8 (6-11) | 1 (0-1) |
| median (IQR) | | | |
| Ventilation: | | | |
| Respirator/NIV/ | 9/3/3 | 11/0/4 | 0/0/15 |
| spontaneous | | | |
| Steroid (Yes/No) | 8/7 | 0/15 | 0/15 |
| Euglycaemic clamp | 9/6 | 1/14 | 0/15 |
| (yes/no) | | | |
| Dialysis (yes/no) | 2/13 | 0/15 | 0/15 |
| Inotropi | | | |
| Nihil | 4 | 5 | 15 |
| Noradrenaline | 4 | 9 | 0 |
| Dopamin | 0 | 0 | 0 |
| Adrenalin | 0 | 0 | 0 |
| Metaoxidrin | 0 | 1 | 0 |
| >1 agent | 6 | 0 | 0 |
| >2 agents | 1 | 0 | 0 |
| Antibiotics | | | |
| Nihil | 0 | 10 | 15 |
| monotherapy | 1 | 3 | 0 |
| polytherapy | 14 | 2 | 0 |
| Nutrition: | | | |
| Nihil/enteral | 10/3/2 | 6/9/0 | 15/0/0 |
| nutrition/TPN | | | |
| Smoking (yes/no) | 9/6 | 3/12 | 0/15 |
| Alcohol abuse | 5/10 | 1/14 | 0/15 |
| (yes/no) | | | |
| Sampletime (a.m.), | 10 (9-11) | 10 (9.5-11) | 9 (9-10) |
| median (IQR) | | | |

Data are given as median with Inter-quartile range (IQR).
Temp Temperature,
HR Heart rate,
RR Respiratory rate,
WBC white blood cell,
MAP Mean arterial pressure,
APACHEII Acute Physiology And Chronic Health Evaluation II,
SOFA Sequential Organ Failure Assessment,
NIV Non-invasive ventilation,
TPN Total parenteral nutrition,
a.m. Ante meridiem.

TABLE 4
Expression of monocyte-bound CD163 and CD206 and levels of sCD163
and sCD206 and laboratory values for the study population.

| | | Sepsis | | Non-sepsis | | Control |
|---|---|---|---|---|---|---|
| | day | n = 15 (21) | | n = 15 | | n = 15 |
| CD163 (MFI) | 1 | 931 | (547-1554) | 449 | (286-661) | 276 (227-355) |
| | 2 | 1104 | (292-1346) | 294 | (233-487) | — |
| | 3 | 931 | (685-1104) | 229 | (184-437) | — |
| | 4 | 533 | (334-874) | 183 | (85-276) | — |
| sCD163 (mg/ml) | 1 | 3.70 | (2.19-11-18) | 1.31 | (0.94-1.48 | 1.35 (1.00-1.60) |
| | 2 | 5.04 | (2.09-11.21) | 1.36 | (1.00-1.61) | — |
| | 3 | 4.98 | (2.62-14.87) | 1.34 | (1.01-1.65) | — |
| | 4 | 5.54 | (2.95-14.27) | 1.58 | (1.08-1.88) | — |
| CD206 (MFI) | 1 | 14.9 | (11.9-19.8) | 11.2 | (10.1-12.4) | 11.5 (10.2-12.5) |
| | 2 | 14.7 | (13.8-17.0) | 10.9 | (10.7-11.5) | — |
| | 3 | 13.5 | (11.9-16.4) | 11.3 | (10-12.4) | — |
| | 4 | 12.1 | (10.6-14.7) | 10.6 | (9.8-12.7) | — |
| sCD206 (mg/ml) | 1 | 1.11 | (0.87-1.94) | 0.35 | (0.29-0.46) | 0.24 (0.2-0.33) |
| | 2 | 1.22 | (0.98-1.98) | 0.47 | (0.31-0.55) | — |
| | 3 | 1.39 | (1.26-2.15) | 0.51 | (0.37-0.66) | — |
| | 4 | 1.52 | (1.38-2.29) | 0.56 | (0.35-0.71) | — |
| APACHE II score | 1 | 17 | (15-22) | 18 | (15-23) | 5 (5-6) |
| SOFA score | 1 | 8 | (7-12) | 8 | (6-11) | 1 (0-1) |
| | 2 | 9 | (7-12) | 9 | (6-11) | — |
| | 3 | 7 | (5-12) | 8 | (4-10) | — |
| | 4 | 8 | (5-12) | 7 | (3-9) | — |
| WBC (×10⁹/l) | 1 | 12.5 | (8.4-21.5) | 12.1 | (10.5-14.1) | 5.1 (4.5-6.6) |
| | 2 | 13.5 | (8.6-21.2) | 11.1 | (8.1-12.9) | — |
| | 3 | 17.1 | (10.7-18.5) | 9.7 | (7.7-11.6) | — |
| | 4 | 14.7 | (12.5-18.2) | 9.4 | (6.5-10.9) | — |
| Lactate (mmol/l) | 1 | 1.9 | (1.5-4.5) | 1 | (0.7-1.2) | 1.3 (1-2) |
| | 2 | 2.1 | (1.5-3.2) | 0.9 | (0.7-13) | — |
| | 3 | 1.9 | (1.3-3.6) | 1.2 | (0.9-1.4) | — |
| | 4 | 2 | (1.5-2.9) | 1.15 | (0.9-1.3) | — |
| CRP (mg/l) | 1 | 139.3 | (92.2-248.8) | 47.15 | (24.7-89) | 0 (0-0.7) |
| | 2 | 202.7 | (117.6-346.9) | 88.1 | (32.8-159.8) | — |
| | 3 | 151.9 | (111.1-308.8) | 84.2 | (33.1-138.5) | — |
| | 4 | 74.8 | (61.4-166.7) | 65.2 | (16.4-117.9) | — |

Data are given as median with Inter-quartile range (IQR).
APACHEII Acute Physiology And Chronic Health Evaluation II,
SOFA Sequential Organ Failure Assessment,
WBC white blood cell,
CRP C-reactive protein

TABLE 5
Area under the receiver operating characteristic curves
for severe sepsis and septic shock and mortality

| | | Outcome | | | | | | | |
|---|---|---|---|---|---|---|---|---|---|
| | | Severe sepsis and | | Mortality | | | | | |
| | | septic shock | | In-hospital | | 28 day | | 90 day | |
| | day | AUC | 95% CI | AUC | 95% CI | AUC | 95% CI | AUC | 95% CI |
| CD163 | 1 | 0.73 | 0.56 to | 0.77 | 0.60 to | 0.80 | 0.64 to | 0.77 | 0.60 to |
| | | | 0.90 | | 0.95 | | 0.96 | | 0.95 |
| | 2 | 0.71 | 0.54 to | 0.83 | 0.67 to | 0.84 | 0.71 to | 0.83 | 0.67 to |
| | | | 0.89 | | 0.98 | | 0.98 | | 0.98 |
| | 3 | 0.89 | 0.77 to | 0.85 | 0.73 to | 0.83 | 0.69 to | 0.85 | 0.73 to |
| | | | 1.00 | | 0.98 | | 0.96 | | 0.98 |
| | 4 | 0.83 | 0.69 to | 0.80 | 0.66 to | 0.78 | 0.62 to | 0.80 | 0.66 to |
| | | | 0.98 | | 0.95 | | 0.94 | | 0.95 |
| sCD163 | 1 | 0.95 | 0.88 to | 0.78 | 0.60 to | 0.68 | 0.48 to | 0.78 | 0.60 to |
| | | | 1.00 | | 0.95 | | 0.89 | | 0.95 |
| | 2 | 0.92 | 0.82 to | 0.77 | 0.59 to | 0.68 | 0.46 to | 0.77 | 0.59 to |
| | | | 1.00 | | 0.95 | | 0.89 | | 0.95 |
| | 3 | 0.92 | 0.83 to | 0.80 | 0.63 to | 0.71 | 0.50 to | 0.80 | 0.63 to |
| | | | 1.00 | | 0.98 | | 0.92 | | 0.98 |
| | 4 | 0.95 | 0.88 to | 0.84 | 0.69 to | 0.76 | 0.57 to | 0.84 | 0.69 to |
| | | | 1.00 | | 0.98 | | 0.95 | | 0.98 |
| CD206 | 1 | 0.86 | 0.73 to | 0.79 | 0.64 to | 0.76 | 0.61 to | 0.79 | 0.64 to |
| | | | 0.98 | | 0.95 | | 0.92 | | 0.95 |
| | 2 | 0.85 | 0.71 to | 0.81 | 0.66 to | 0.79 | 0.64 to | 0.81 | 0.66 to |
| | | | 0.98 | | 0.95 | | 0.94 | | 0.95 |
| | 3 | 0.78 | 0.63 to | 0.72 | 0.56 to | 0.70 | 0.52 to | 0.72 | 0.56 to |
| | | | 0.94 | | 0.89 | | 0.87 | | 0.89 |
| | 4 | 0.64 | 0.45 to | 0.54 | 0.34 to | 0.57 | 0.34 to | 0.54 | 0.34 to |
| | | | 0.83 | | 0.75 | | 0.79 | | 0.75 |
| sCD206 | 1 | 1.00 | 1.00 to | 0.87 | 0.74 to | 0.76 | 0.59 to | 0.87 | 0.74 to |
| | | | 1.00 | | 0.99 | | 0.93 | | 0.99 |
| | 2 | 1.00 | 1.00 to | 0.88 | 0.75 to | 0.77 | 0.61 to | 0.88 | 0.75 to |
| | | | 1.00 | | 1.00 | | 0.94 | | 1.00 |
| | 3 | 1.00 | 1.00 to | 0.90 | 0.79 to | 0.80 | 0.65 to | 0.90 | 0.79 to |
| | | | 1.00 | | 1.00 | | 0.96 | | 1.00 |
| | 4 | 0.99 | 0.98 to | 0.93 | 0.84 to | 0.84 | 0.69 to | 0.93 | 0.84 to |
| | | | 1.00 | | 1.00 | | 0.98 | | 1.00 |

### Example 4

### Increased Concentrations of Soluble Mannose Receptor in Serum from Patients with Pneumococcal Bacteremia and Prediction of Survival

Streptococcus pneumoniae (S. pneumoniae) is one of the most frequent causative agents of death from infections in both Europe and the US. It is estimated that 1.6 million people world-wide die of pneumococcal disease every year. When S. pneumonia is cultured from blood or other sterile sites, the infection is defined as invasive pneumococcal disease (IPD), which has a mortality of 10-25% even when the correct antibiotic treatment is administered.

Materials and Methods

The patients were enrolled from 5 university hospitals in Denmark between October 1999 and June 2001. Results from the study have been published previously [Møller et al (2006). Crit Care Med 34(10):2561-6; Kronborg et al (2002). J Infect Dis 15; 185(10):1517-20; Kronborg et al (2002). Scand J Infect Dis 34(5):323-6; Wittenhagen et al (2004). Clin Microbiol Infect. 2004 May; 10(5):409-15; Kronborg G (2004). Ugeskr Laeger 24; 166(22):2132]. The present example is based on measurements of the soluble form of the mannose receptor in human serum.

Study Design and Patients

The initial inclusion criteria were adults (>17 years old) with fever (>38° C.) or infection suspected by the clinical presentation of the patient. If blood cultures were positive for S. pneumoniae within 12-36 hours, then blood samples were drawn within the next 24 hours for later analysis of macrophage biomarkers. Serum was stored at −20° C. until biochemical analyses were performed.

Biochemical Analyses

Serum sMR was analyzed in duplicate samples of frozen serum as described in example 1. CD163 and CRP were measured as described in a previous publication [Møller et al 2006 op cit].

Ethics

The study was approved by the local ethical committees.

Statistics

sMR values were normally distributed after log-transformation. sCD163 was normally distributed without transformation. CRP values did not follow a normal distribution neither with nor without log-transformation.

Spearman's test was used for correlation analysis of non-parametric data, and

Pearson's correlation analysis was used for parametric data.

Students T-test was used for analysis of between groups difference.

Receiver operator characteristic analyses (ROC curves) were applied for analysis of the prognostic performance of the biomarkers.

Results

In total, 141 patients with culture results positive for S. pneumoniae were included. Serum was accessible for sCD163 analysis from 133 of the patients, but corresponding serum samples for sMR analysis were only available from 128 patients. The median age of the 133 patients was 66 years, with a range of 23-99 yrs. 95 patients were under 75 years of age (48 men and 47 women with median ages of 58 and 62 years, respectively). 11 men and 27 women were 75 years or above (median age 84 and 83 years, respectively).

Pneumonia was the most common focus on infection (n=110). Twenty-three patients died in the hospital (case fatality rate, 17%). An underlying chronic illness was present in 65% of the patients. All patients received adequate antibiotic treatment (e.g., penicillin or cephalosporin) within a few hours of admission and before the result of the positive blood culture was available.

Correlations between sMR serum concentrations and sCD163, and routine biochemical markers are shown in table 6.

The median sMR concentration in the entire group of patients (n=128) was 0.77 mg/L (CI: 0.70-0.90 mg/L), which is significantly above the upper reference range for sMR in healthy individuals (0.1-0.43 mg/L). In patients under the age of 75 (n=92) the median sMR concentration was 0.82 mg/L (CI: 0.71-0.97 mg/L). In patients 75 years or older (n=36) the median sMR concentration was 0.73 mg/L (CI: 0.56-0.86).

For the entire group (n=128) there was a significant difference in the sMR concentration between survivors (n=107) and non-survivors (n=21), (p<0.001) (FIG. 11A). For the patients below the age of 75 (n=92) there was also a significant difference in the sMR concentration between survivors (n=76) and non-survivors (n=16) (p<0.001) (FIG. 11B). In patients 75 years or older (n=36) the difference in sMR concentrations between survivors (n=31) and non-survivors (n=5) was in-significant (FIG. 11C).

In ROC curve analysis for the prediction of survival in the entire group of patients (n=128) sMR had the highest (sMR AUC=0.79, sCD163 AUC=0.70, CRP AUC=0.73) (FIG. 12A). When looking at the patient under the age of 75 years (n=90), AUC was higher for both sMR (0.86), sCD163 (0.81), and CRP (0.76) (FIG. 12B). For the patients 75 years or older the AUC were lower than for the entire group of patients for all three markers (sMR=0.56, sCD163=0.38, CRP=0.66) (FIG. 12C).

TABLE 6
Correlations between sMR and sCD163, CRP, and
routine biochemical parameters.

| | R | p |
|---|---|---|
| Leucocytes | −0.21 | 0.02 |
| Neutrofiles | −0.23 | 0.04 |
| CRP | 0.36 | <0.001 |
| sCD163 | 0.62 | <0.001 |

### CONCLUSION

The present study shows that for IPD patients under the age of 75, serum sMR within 24 hours after the time of positive blood culture is significantly higher in patients who subsequently die from the disease compared to those who survive. Furthermore, sMR is superior to both sCD163 and CRP for the prognosis of a fatal outcome in IPD patients.

### Example 5

### Use of Soluble Mannose Receptor to Differentiate Mild Liver Fibrosis from Cirrhosis in Patients with Chronic Hepatitis

Methods

Patients and Samples Collection

Patients chronically infected with HCV [HCV-RNA detectable by polymerase chain reaction (PCR) in blood≧6 months], genotype 1, were included in the study if they had either no/mild fibrosis or cirrhosis, as determined from a successful examination with transient elastography (TE). Patients were included between January 2009 and March 2010 from two Danish hospital clinics specialised in hepatitis, Department of Infectious

Diseases, Copenhagen University Hospital, Hvidovre, and Department of Infectious Diseases, Odense University Hospital. At the day of examination with TE, blood samples were drawn for HCV-RNA detection, platelet counts and analysis of alanine amino-transferase (ALT), alkaline phosphatase, bilirubin levels, albumin concentration and clotting factors II, VII and X. Creatinine concentrations for the calculation of Model for End-Stage Liver Disease (MELD) scores were measured on the day of examination with TE for most of the patients, but for 11 patients, measurements were taken up to one year before or after the date of examination. Furthermore, at the day of examination with TE, a plasma sample was centrifuged and stored at −20° C. for later analysis for sCD163, sMR and other potential markers. The results from the measurements of 12 fibrosis markers including TNF-α and suPAR have previously been published [Andersen et al (2011). Eur J Clin Microbiol Infect Dis 30:761-766]. Based on power considerations for expected differences between the two groups, we planned to include 40 participants.

Inclusion Criteria

Patients older than 18 years of age, chronically infected with HCV, genotype 1, and with no/mild liver fibrosis (liver stiffness measurement<7.7 kPa) or cirrhosis (liver stiffness measurement≧13.0 kPa) were offered participation in the study.

Exclusion Criteria

Patients infected with HCV non-genotype 1, with hepatitis B virus or with human immunodeficiency virus (HIV) were not included in the study. Furthermore, patients were excluded if they had been administered interferon or ribavirin up to one year before examination. Patients with hepatocellular carcinoma, previous liver transplantation or liver metastases were not included in the study. Patients with a self-reported daily alcohol consumption of >36 g for females and >60 g for males were not included.

Transient Elastography

We used FibroScan® (Echosens, Paris, France) with a medium probe and software version 1.30 for the liver stiffness measurements. The examination was performed by two physicians certified in the use of FibroScan® and with experience of more than 300 examinations. Liver stiffness measurements were considered successful if ten valid measurements were obtained (success rate of valid measurements>60%). The interquartile range of the measurements should be less than 25% of the median value. Patients were considered as having no/mild fibrosis when the liver stiffness was below 7.7 kPa and as having cirrhosis when the liver stiffness was equal to or above 13.0 kPa. These cut-off values had been selected prior to the study and are based on a large meta-analysis including 50 studies of correlation between fibrosis determined by liver biopsy and TE [Friedrich-Rust et al (2008). Gastroenterology 134:960-974]. For diagnosing cirrhosis with a cut-off value of 13.0 kPa using liver biopsies as the reference, a mean area under the receiver operating characteristic curve (AUC) of 0.94 has been found and for diagnosing significant fibrosis (F2 in the METAVIR classification) with liver stiffness above 7.7 kPa, the mean AUC was 0.84 [Friedrich-Rust et al 2008 op cit].

Biochemical Analysis

Blood samples were drawn from patients on the same day that the liver stiffness measurements were carried out. Blood from one 9-ml EDTA-coated tube was separated by centrifugation and plasma was stored at −20° C. Serum sCD163 and sMR were analysed as in example 1.

Statistics

Plasma concentrations of sCD163 and sMR for patients with no/mild fibrosis and cirrhosis were compared using non-parametric tests. The diagnostic performance of the plasma proteins was assessed using receiver operating characteristic (ROC) curve analysis and compared in accordance with the method suggested by Hanley and McNeil [1983, Radiol 148:839-843]. The software R Statistics, version 2.9.0 (R Development Core Team, Vienna, Austria) and SPSS version 17.0 (SPSS Inc., Chicago, Ill., USA) were used for the analyses. Correlation analyses (Pearson r) were performed using GraphPad Prism version 4.00 for Windows (GraphPad Software, San Diego, Calif., USA). p-values<0.05 were considered significant.

Ethics

The Danish National Committee on Biomedical Research Ethics (H-D-2007-0087) approved the study in accordance with the Helsinki Declaration. All patients gave written consent to participate in the study.

Results

The characteristics of the 40 patients with hepatitis C, genotype 1, and either no/mild fibrosis (19 patients) or cirrhosis (21 patients) are shown in Table 7. We had information on the presence of ascites for 37 of the 40 patients included. Only one of the patients (in the cirrhosis group) had ascites. All of the patients were followed in out-patient clinics and had no signs of encephalopathy. The Child-Pugh class and MELD scores (available for 37 patients) are presented in Table 7.

The plasma concentrations of sCD163 and sMR were compared between patients with no/mild fibrosis and cirrhosis. Both sCD163 and sMR were significantly higher in cirrhotic patients than in patients with no/mild fibrosis (Table 8, FIG. 13a, b) and the plasma concentrations of the two markers correlated significantly (FIG. 13c). In the ROC analyses for the diagnosis of cirrhosis, the AUC for sCD163 was higher than that for sMR (FIG. 13d), although the difference was not significant (Table 8).

The correlations between sCD163 and sMR and other immunological markers are shown in Table 9. Generally, sCD163 and sMR show the same pattern of positive and negative associations.

TABLE 7
Characteristics of the 40 patients with chronic hepatitis C,
genotype 1, and either no/mild fibrosis or cirrhosis

| | No/mild fibrosis | | Cirrhosis | | p value^{h} |
|---|---|---|---|---|---|
| Patients^{a} | 19 | (47.5%) | 21 | (52.5%) | |
| Gender (males)^{a} | 8 | (42%) | 15 | (71%) | 0.117 |
| Age (years)^{b} | 46 | (38; 54) | 57 | (51; 61) | 0.001 |
| BMI (kg/m2)^{b} | 22.6 | (20.4; 26.1) | 26.5 | (23.9; 29.9) | 0.019 |
| Liver stiffness | 4.9 | (4.7; 6.1) | 23.2 | (16.9; 32.3) | |
| (kPa)^{b} | | | | | |
| Alk phosph (U/l)^{b,c} | 82 | (69.5; 87.3) | 95 | (73.5; 126.5) | 0.020 |
| Factors II, VII, | 0.94 | (0.70; 1.04) | 0.71 | (0.60; 0.87) | 0.020 |
| X (U/l)^{b,d} | | | | | |
| ALT (IU/l)^{b,e} | 38 | (27; 59) | 94 | (52; 144) | <0.001 |
| Platelets (10⁹/l)^{b} | 242 | (188; 260) | 144 | (107; 205) | <0.001 |
| Bilirubin (μmol/l) | 7 | (4; 8) | 11 | (11; 15.5) | 0.003 |
| MELD scores^{f} | −0.60 | (−2.47; 3.60) | 3.95 | (1.9; 5.50) | 0.006 |
| Child-Pugh | | | | | |
| classification | | | | | |
| A^{a} | 17 | (100%) | 19 | (95%) | |
| B^{a} | 0 | (0%) | 1 | (5%) | |
| C^{a} | 0 | (%) | 0 | (0%) | |
| NA^{a,g} | 2 | (10.5%) | 1 | (4.8%) | |

^{a}Number of patients (%)
^{b}Median with 25th and 75th percentiles in parentheses
^{c}Alkaline phosphatase
^{d}Clotting factors II, VII and X
^{e}Alanine aminotransferase
^{f}Model for End-Stage Liver Disease
^{g}Not applicable
^{h}Mann-Whitney test for equality

TABLE 8
Median plasma concentrations of soluble CD163 (sCD163) and soluble
mannose receptor (sMR) for the 40 patients with chronic hepatitis C,
genotype 1, and either no/mild fibrosis or cirrhosis
Serum markers

| | Liver fibrosis | | | |
|---|---|---|---|---|
| | No/mild fibrosis | Cirrhosis | p-value^{a} | AUC^{b} |
| sCD163 | 2.49 (1.90; 3.49) | 5.77 (4.51; 8.39) | <0.001 | 0.89 |
| | | | | (0.79-0.99) |
| sMR | 0.30 (0.22; 0.40) | 0.44 (0.30; 0.64) | 0.005 | 0.75 |
| | | | | (0.61-0.90) |

25th and 75th percentiles are shown in parentheses
^{a}Mann-Whitney test for equality
^{b}Area under the receiver operating characteristic curve (95% confidence interval, CI)

TABLE 9
Correlation between soluble CD163 (sCD163) and soluble mannose
receptor (sMR) and earlier described biochemical parameters or
markers (Pearson r analyses) and Model for End-Stage Liver
Disease (MELD) score

| | |
|---|---|
| sCD163 | sMR |

| | r2 | p-value | r2 | p-value |
|---|---|---|---|---|
| Direct immunological | | | | |
| markers | | | | |
| TNF-α^{a} | 0.40 | <0.001 | 0.20 | 0.004 |
| IL1-β^{b} | −0.01 | NS | 0.00 | NS |
| IL6^{c} | −0.03 | NS | 0.01 | NS |
| IL8^{d} | 0.08 | NS | 0.18 | 0.006 |
| MCP-1^{e} | 0.15 | 0.013 | 0.14 | 0.016 |
| suPAR^{f} | 0.68 | <0.001 | 0.32 | <0.001 |
| MIG^{g} | 0.38 | <0.001 | 0.26 | <0.001 |
| Indirect markers | | | | |
| ALT^{h} | 0.15 | 0.016 | 0.08 | NS |
| Platelets | −0.23 | 0.002 | −0.12 | 0.032 |
| Alk phosphⁱ | 0.15 | 0.016 | 0.16 | 0.013 |
| Factors II, VII and X^{j} | −0.17 | 0.013 | −0.11 | 0.050 |
| Liver status | | | | |
| MELD scores^{k} | 0.32 | <0.001 | 0.13 | 0.026 |

^{a}Tumour necrosis factor-α
^{b}Interleukin 1-β
^{c}Interleukin 6
^{d}Interleukin 8
^{e}Monocyte chemotactic protein-1
^{f}Soluble urokinase-type plasminogen activator
^{g}Monokine induced by γ-interferon
^{h}Alanine aminotransferase
ⁱAlkaline phosphatase
^{j}Clotting factors II, VII, and X
^{k}Model for End-Stage Liver Disease

1. A method for assessing the likelihood that a subject suffers from a disorder and/or disease, said method comprising determining the amount of soluble CD206 in a biological sample from said subject wherein a high level of soluble CD206 is indicative of an increased likelihood.

2. A method for assessing the severity of a disorder in a subject diagnosed with said disorder, the method comprising determining the amount of soluble CD206 in a biological sample from said subject wherein a high level of soluble CD206 is indicative of an increased severity.

3. A method of prognosing or diagnosing disease progression in a subject diagnosed with said disease, the method comprising determining the amount of soluble CD206 in a biological sample from said subject wherein a high level of soluble CD206 is indicative of disease progression.

4. The method of claim 2, wherein an indication of increased severity comprises prediction of fatal outcome.

5. The method of any of the preceding claims, wherein said disorder is selected from the group consisting of endocrinological disorders, haematological disorders, and hepatological disorders.

6. The method of any of the preceding claims, wherein the disorder is an infection.

7. The method of claim 6, wherein the disorder is a bacterial and/or viral infection.

8. The method of any of the preceding claims, wherein the disorder is sepsis.

9. The method of claim 8, wherein the sepsis is caused by a bacterial infection.

10. The method of claim 8, wherein the bacterial infection is caused by a Gram-positive bacteria selected from the group consisting of: Streptococcus pneumonia and Streptococcus pyogenes.

11. The method of claim 5, wherein the hepatological disorder is selected from the group consisting of alcoholic liver disease, toxic liver disease, autoimmune hepatitis, viral hepatitis, non-alcoholic fatty liver disease, liver-cirrhosis, liver failure

12. The method of claim 5, wherein the endocrinological disorder is diabetes.

13. The method of claim 1, wherein said disorder or disease is a fungal infection and/or a fungal associated disorder or disease.

14. The method of claim 13, wherein said disorder or disease is a complication associated with fungal infection.

15. The method of claims 13, wherein the fungal infection is caused by Candida, Aspergillus, Cryptococcus, Histoplasma, Pneumocystis, and Stachybotrys.

16. The method of any of the preceding claims, wherein said biological sample is selected from the group consisting of serum, plasma, or whole blood.

17. The method of any of the preceding claims, wherein determination of the amount of CD206 comprises a binding assay, such as mannose binding assay, or an antibody based quantitative assessment.

18. The method of any of the preceding claims, wherein determination of the amount of CD206 comprises a liquid chromatographic method.

19. The method of any of the preceding claims, wherein determination of the amount of CD206 comprises mass spectrometry.

20. The method of any of the preceding claims, wherein determination of the amount of soluble CD206 comprises turbidometry or nephelometry.

21. The method of any of the preceding claims, wherein a high level of sCD206 comprises a value higher than the 60^{th} percentile, for example higher than the 65^{th} percentile, such as higher than the 67^{th} percentile, for example higher than the 70^{th} percentile, such as higher than the 75^{th} percentile, for example higher than the 80^{th} percentile, such as higher than the 85^{th} percentile, for example higher than the 90^{th} percentile, such as higher than the 95^{th} percentile, for example higher than the 97^{th} percentile.

22. The method of any of the claims 1-17, wherein a high level of sCD206 comprises a value higher than 0.9×97.5^{th} percentile, such as higher than 1.0×97.5^{th} percentile, for example 1.1×97.5^{th} percentile, such as 1.2×97.5^{th} percentile, for example 1.3×97.5^{th} percentile, such as 1.4×97.5^{th} percentile, for example 1.5×97.5^{th} percentile, such as 1.6×97.5^{th} percentile, for example 1.7×97.5^{th} percentile, such as 1.8×97.5^{th} percentile, for example 1.9×97.5^{th} percentile, such as 2.0×97.5^{th} percentile, for example 2.1×97.5^{th} percentile, such as 2.2×97.5^{th} percentile, for example 2.3×97.5^{th} percentile, such as 2.4×97.5^{th} percentile, for example 2.5×97.5^{th} percentile.

23. The method of claims 17-18, wherein said percentile is determined for subset of individuals, said individuals having the same gender, race, or belonging to group based on age, BMI, smoking habit, occupation, physical inactivity, hip circumference, waist circumference, systolic and/or diastolic blood pressure, alcohol consumption, a combination of any subset of these, or other risk factor.

24. The method according to any of the preceding claims, wherein a subject is considered to suffer from a disorder and/or disease, if the measured amount of soluble CD206 in said biological sample is higher than the amount found in the reference group.

25. The method of claim 20, wherein the reference group is the group constituting the 0-33^{th} percentile for sCD206.

26. The method of any of the preceding claims, wherein said assessment comprises comparing the level of sCD206 to a predetermined cutoff level, wherein a level of sCD206 above the cutoff level is indicative of an increased likelihood, severity, disease progression, or fatal outcome.

27. The method of claim 22, wherein said cutoff level is a level measured in the same subject, when the subject was healthy.

28. The method of claim 22, wherein said cutoff level is a level is an average of measurement made in a large group of healthy subjects, optionally grouped according to age, gender, race, or population group.

29. The method of claim 22, wherein said cutoff level is at least 0.25 mg/L sCD206, such as at least 0.3 mg/L, for example at least 0.35 mg/L, such as at least 0.4 mg/L, for example at least 0.43 mg/L, such as at least 0.5 mg/L.

30. The method of claim 22, wherein said cutoff level is at least 1.5× a median level of sCD206 for healthy individuals, such as 1.75, for example 2.0, such as 2.25, for example 2.5, such as 2.75, for example 3.0, such as 3.25, for example 3.5.

31. The method of any of the preceding claims, wherein the subject is a Caucasian.

32. The method of claim 1, wherein the subject appears to be healthy.

33. The method of claim 1, wherein the subject appears unwell but has not been diagnosed with a specific disorder.

34. The method of any of the preceding claims 1, wherein the subject is ill, an optionally has been diagnosed.

35. The method of any of the preceding claims, wherein said assessment further comprises determining at least one further biochemical parameter, preferably wherein said further biochemical parameter is selected from the group consisting of lactate, creatinine, blood glucose, inflammatory markers such as CRP, Fibrinogen, alpha1-antitrypsin, liver parameters such as ALAT, gammaGT, alkaline phosphatise, lactate dehydrogenase, bilirubine, and soluble CD163.

36. The method of any of the preceding claims, further comprising assessing at least one further risk factor such as gender, race, age, BMI, weight, smoking habit, physical inactivity, hip circumference, waist circumference, systolic and/or diastolic blood pressure, alcohol consumption.

37. A method of treatment of a disease or disorder said method comprising assessing the level of soluble CD206 and subsequently treating said disease or disorder if the level of CD206 is above a pre-defined cutoff level.

38. The method of claim 37, wherein the disorder is selected from endocrinological disorders, haematological disorders, and hepatological disorders.

39. The method of claim 37, wherein the disorder is pneumonia.

40. The method of claim 37, wherein the disorder is sepsis.

41. A method of assessing the efficacy of a therapy given to a patient, said method comprising assessing the level of soluble CD206 in serum from said patient, subjecting said patient to therapy, subsequently assessing the level of soluble CD206 again, wherein said therapy is efficacious if the level of soluble CD206 decreases.

42. The method of claim 41, wherein the patient suffers from a disorder selected from endocrinological disorders, haematological disorders, hepatological disorders, and liver disorders.

43. The method of claim 41 or 42, wherein the disorder is pneumonia

44. The method of claim 41 or 42, wherein the disorder is sepsis.

45. The method of any of claims 41 to 44, wherein the therapy is experimental, such as wherein the therapy is part of a clinical trial.

46. The method of any of claims 41 to 45, wherein said therapy is discontinued if the level of sCD206 increases.

47. The method of any of claims 41 to 46, wherein said therapy is replaced with a different therapy if the level of sCD206 increases.

48. A method of assessing the safety of a therapy, comprising determining the level of soluble CD206 in a subject, subjecting said subject to said therapy, and determining the level of soluble CD206 after the therapy, wherein the absence of a significant increase in soluble CD206 indicates that the therapy is safe.

49. The method of claim 48, wherein said therapy is safe for the liver.

50. A method of screening donor blood, comprising determining the level of soluble CD206 in a sample of donor blood, wherein said donor blood is discarded if the level of sCD206 is above a predetermined cutoff level.

51. Use of an agent for the manufacture of a diagnostic agent for diagnosing, monitoring or determining the likelihood of a disease or disorder in a human being, wherein said use discriminates between a disease or disorder and healthy condition, wherein the diagnostic agent does this by:
i) determining the concentration of human soluble CD206 in a sample of urine, plasma or serum from the human being,
ii) comparing said concentration with a predetermined cutoff value, chosen to exclude lower concentrations of soluble CD206 associated with a healthy condition, wherein a concentration above the cutoff value is indicative of a disorder.

52. A method comprising:
i) determining the concentration of soluble CD206 of a sample of bodily fluid taken from the human body;
ii) indicating whether the soluble CD206 concentration determined in step i) deviates from a predetermined cutoff value.


SEQUENCE LISTING (5 sequences)

SEQ ID NO: 1 - protein, 1456 aa, Homo sapiens [sequence omitted]

SEQ ID NO: 2 - protein, 1456 aa, Mus musculus
MRLLLLLAFISVIPVSVQLLDARQFLIYNEDHKRCVDALSAISVQTATCNPEAESQKFRWVSDSQIMSVAFKLCLGVPSKTDWASVTLYACDSKSEYQKWECKNDTLFGIKGTELYFNYGNRQEKNIKLYKGSGLWSRWKVYGTTDDLCSRGYEAMYSLLGNANGAVCAFPFKFENKWYADCTSAGRSDGWLWCGTTTDYDKDKLFGFCPLHFEGSERLWNKDPLTGILYQINSKSALTWHQARASCKQQNADLLSVTEIHEQMYLTGLTSSLSSGLWIGLNSLSVRSGWQWAGGSPFRYLNWLPGSPSSEPGKSCVSLNPGKNAKWENLECVQKLGYICKKGNNTLNPFIIPSASDVPTGCPNQWWPYAGHCYRIHREEKKIQKYALQACRKEGGDLASIHSIEEFDFIFSQLGYEPNDELWIGLNDIKIQMYFEWSDGTPVTFTKWLPGEPSHENNRQEDCVVMKGKDGYWADRACEQPLGYICKMVSQSHAVVPEGADKGCRKGWKRHGFYCYLIGSTLSTFTDANHTCTNEKAYLTTVEDRYEQAFLTSLVGLRPEKYFWTGLSDVQNKGTFRWTVDEQVQFTHWNADMPGRKAGCVAMKTGVAGGLWDVLSCEEKAKFVCKHWAEGVTRPPEPTTTPEPKCPENWGTTSKTSMCFKLYAKGKHEKKTWFESRDFCKAIGGELASIKSKDEQQVIWRLITSSGSYHELFWLGLTYGSPSEGFTWSDGSPVSYENWAYGEPNNYQNVEYCGELKGDPGMSWNDINCEHLNNWICQIQKGKTLLPEPTPAPQDNPPVTADGWVIYKDYQYYFSKEKETMDNARAFCKKNFGDLATIKSESEKKFLWKYINKNGGQSPYFIGMLISMDKKFIWMDGSKVDFVAWATGEPNFANDDENCVTMYTNSGFWNDINCGYPNNFICQRHNSSINATAMPTTPTTPGGCKEGWHLYKNKCFKIFGFANEEKKSWQDARQACKGLKGNLVSIENAQEQAFVTYHMRDSTFNAWTGLNDINAEHMFLWTAGQGVHYTNWGKGYPGGRRSSLSYEDADCVVVIGGNSREAGTWMDDTCDSKQGYICQTQTDPSLPVSPTTTPKDGFVTYGKSSYSLMKLKLPWHEAETYCKDHTSLLASILDPYSNAFAWMKMHPFNVPIWIALNSNLTNNEYTWTDRWRVRYTNWGADEPKLKSACVYMDVDGYWRTSYCNESFYFLCKKSDEIPATEPPQLPGKCPESEQTAWIPFYGHCYYFESSFTRSWGQASLECLRMGASLVSIETAAESSFLSYRVEPLKSKTNFWIGMFRNVEGKWLWLNDNPVSFVNWKTGDPSGERNDCVVLASSSGLWNNIHCSSYKGFICKMPKIIDPVTTHSSITTKADQRKMDPQPKGSSKAAGVVTVVLLIVIGAGVAAYFFYKKRHALHIPQEATFENTLYFNSNLSPGTSDTKDLMGNIEQNEHAII

SEQ ID NO: 3 - protein, 1463 aa, Rattus norvegicus
MEHTLWAMRLPLLLAFISVIPVAVQLLDSRQFLIYNEDHKRCVDALSAISVQTATCNPEAESQKFRWVSESQIMSVAFKLCLGVPSKTDWASVTLYACDSKSEFQKWECKNDTLFGIKGTELYFNYGNRQEKNIKLYKGSGLWSRWKVYGTTDDLCSRGYEAMYSLLGNANGAVCAFPFKFENKWYADCTSAGRSDGWLWCGTTTDYDTDKLFGFCPLQFEGSKRLWNTDPLTGILYQINSKSALTWHQARASCKQQNAELLSVTEIHEQMYLTGLTSSLTSGLWIGLNSLSLSTGWQWAGGSPFRYLNWLPGSPSSEPGKSCVSLNPGKNAKWENLECVQKLGYICKKGNNTLNPFIIPSESDVPTACPNQWWPYAGHCYRIYREEKKIQKYALQACRKEGGDLASIHSIEEFDFIFSQLGYEPNDELWIGLNDIKIQMYFEWSDGTPVTFTKWLPGEPSHENNRQEDCVVMKGKDGYWADRACEQPLGYICKMVSQIHTVIPEGAEKGCRKGWKRHGFYCYLIGSTLSTFADANQTCTNEKAYLTTVEDRYEQAFLTSLVGLRPEKYFWTGLSDVQNKGTFRWTVDEQVQFTHWNADMPGRKAGCVAMKTGVAGGLWDVLSCEEKAKFVCKHWAEGVTRPPEPTTTPEPKCPEDWGTTSKTSMCFKLYAKGKHEKKTWFESRDFCKAIGGELASIKSKDEQQVIWRLITSSGSYHELFWLGLTYGSPSEGFTWSDGSPVSYENWAYGEPNNYQNVEYCGELKGDPGMSWNDINCEHLNNWICQIQKGKTLLPEPTPAPQDNPPVTADGWVIYKDYQYYFSKEKETMDNARAFCKKNFGDLATIKSESEKKFLWKYINKNGGQSPYFIGLLISLDKKFIWMDGSKVDFVAWATGEPNFANDDENCVTMYTNSGFWNDINCGYPNNFICQRHNSSINATAMPTTPPTPGGCKEGWHLYNNKCFKIFGFAEEEKKTWKEARKACIGLKGNLVSIENAKEQAFVTYHMRDSTFNAWTGLNDVNSEHTFLWTDGRGVHYTNWGKGYPGGRRSSLSYEDVDCVVVIGGNSREAGTWMDSTCDSKQGYICQTQTDPSVPISPTTAPKDGFVKYGKSSYALMKLKSPWHEAEKYCKDRTSLLASILDPYSNAFAWMKMHPFNVPIWIALNSNLTNNEYTWTDKWRVRYTNWGTDEPKLKSACVYMDVDGYWKTSYCNESFYFLCKKSDEIPATEPPQLPGKCPESEQTAWIPFHGHCYYIESSFTRSWGQASLECLRMGASLVSIETAAESSFLSYRVEPLKSKTNFWIGMFRNVEGKWLWLNDNPVSFVNWKTGDPSGERNDCVVLSSSSGLWNNIHCTSYKGFICKMPKIIDPVTTHSSITTKADQRKMDPQPKGSSKAAGVVIVVLLIVIGAGVAAYFFYKKRRVLHIPQEATFENTLYFNSNPSPGTSDTKDLVGNIEQNEHAVI

SEQ ID NO: 4 - protein, 1455 aa, Sus scrofa
MRLSPCLAFLSFLPVALQLLDTRQFLIYNEDHKRCVEALSPSSVQTAVCNQDNEAQKFRWVSESQIMSVAFKLCLGVPSKTDWVPVTLYACDSKSEFQKWECRNDTLLGIKGEDIFFNYGNRQEKNIMLYKGSGLWSRWKVYGTTDDLCSRGYEAMYTLLGNSNGATCAFPFKFENKWYADCTTAGRSDGWLWCGTTTDYDTDKLFGYCPLKFEGIERLWNKDPLTSISYQINSKSALTWHQARKSCQQQNAELLSITEIHEQTYLTGLTSSLTSGLWIGLNSLSFNSGWQWSGGSPFRYLNWLPGSPSAEPGKSCVSLNPGKNAKWENLQCVQKLGYICKKGNTTLNSFVIPSESDVPTSCPSQWWPYAGHCYKIYREEKKIQRDALTACRKEGGDLASIHSIEEFDFIISQLGYEPNDELWIGLNDIKIQMYFEWSDGTPVTFTKWLPGEPSHENNRQEDCAVMKGKDGYWADRACERPLGYICKMKSQAQTPGRVEVETGCRKGWKRHGFYCYLIGHTLSTFAEANQTCENEKAYLTTVEDRYEQAFLTSLVGLRPERYFWTGLSDVQNKGTFQWTIAEGVQFTHWNTDMPGRKAGCVAMRTGVAGGLWDVLRCEEKNKFVCKHWAEGVTRPPEPTTTPEPKCPEDWGTSTKTSLCFKLFAKGKHEKKTWFESRDFCRALGGDLASINNKEEQQAIWRLVTASGSYHELFWLGLTYSSPSEGFTWSDGSPVSYENWAYGEPNNYQNVEYCGELKSDAGMSWNDINCEHLNNWICQIRKGQTPKPEPTPAPQDNPPVTEDGWVIYKDYQYYFSKEKETMDKAREFCKKNFGDLVSIQSESEKKFLWKYVNKNDAQPAYFIGLLISLDKKFIWMDGSKVDYVAWAAGEPNFANDDENCVTMYTHSGFWNDINCGYPNAFICQRHNSSINATVTPTIPSAPGGCKEGWNFYNDKCFKIFGFVEEERKNWQEARKACIGFGGNLASIRNEKEQAFLTYHMKDSTFNAWTGLNDVNSEHTFLWTDGRGVHYTNWGKGYPGGRRSSLSYEDADCVVIIGGKSRDAGKWMDDICDNKRGYICQTLPDSSLPRSPTTIPTDGFIKYGESSYSLTKLKLQWHEAADYCKLHSSLIASILDPYSNAFAWMQMQALSEPVWIALNSNLTNNEYVWTDKWRVRYTNWAADEPRLKTACVYMDLDGSWKTANCNESFYFFCKKSDETPATEPPQLPGRCPESEHTAWIPFHGHCYYIESSYTRNWGQASLECLRMGSSLVSIESAAESSFLSYRVEPLQSKTNFWIGLYRNVEGMWLWVNNNPVSFVNWNTGDPSGERNDCVALYASSGFWNNIHCSSYKGYICKRPKIVDAEPTHALVTTKADPRKMVTSKPSSNSAGVVVIVVLLILTGAGFAAYFFYKKRRVHLPQEENFENTLYFNSASTPGASDTKDLIGNIEQNEHVVI

SEQ ID NO: 5 - protein, 1456 aa, Macaca mulatta
MRLPLLLVFASVIPGAVLLLDTRQFLIYNEDHKRCVEAVSPSAVQTAACNQDAESQKFRWVSESQIMSVAFKLCLGVPSKTDWVAITLYACDSKSEFQKWECKNDTLLGIKGEDLFFNYGNRQEKNVMLYKGSGLWSRWKIYGTTDNLCSRGYEAMYTLLGNANGATCAFPFKFENKWYADCTSAGRSDGWLWCGTTTDYDTDKLFGYCPLKFEGSESLWNKDPLTSISYQINSKSALMWHQARKSCQQQNAELLSITEIHEQTYLTGLTSSLTSGLWIGLNSLSFNSGWQWSDRSPFRYLNWLPGSPSAEPGKSCVSLNPGKNAKWENLECVQKLGYICKKGNTTLNSFVIPSESDVPTHCPSQWWPYAGHCYKIHKDEKKIQRDALTACRKEGGDLASIHTIEEFDFIISQLGYEPNDELWIGLNDIKIQMYFEWSDGTPVTFTKWLRGEPSHENNRQEDCVVMKGKDGYWADRGCEWPLGYICKMKSRSQGPEIVEVEEGCKKGWKKHHFYCYMIGHMLSTFAEANQTCNNENAYLTTIEDRYEQAFLTSFIGLRPEKYFWTGLSDIQTKGTFQWTIEEEVRFTHWNSDMPGRKPGCVAMRTGIAGGLWDVLKCDEKAKFVCKHWAEGVTHPPKPTTTPEPKCPEDWGASSRTSLCFKLYAKGKHEKKTWFESRDFCRALGGDLASINNKEEQQTIWRLITASGSYHELFWLGLTYGSPSEGFTWSDGSPVSYENWAYGEPNNYQNVEYCGELKGDPSMSWNDINCEHLNNWICQIHKGQTPKPEPTPAPQDNPPVTEDGWVIYKDYQYYFSKEKETMDNARAFCKRNFGDLVSIQSESEKKFLWKYVNRNDAQSAYFIGLLISLDKKFAWMDGSKVDYVSWATGEPNFANEDENCVTMYSNSGFWNDINCGYPNAFICQRHNSSINATTVMPTKPSVPSGCKEGWNFYNNKCFKIFGFVEEERKTWQEARKACIGFGGNLVSIQNEKEQAFLTYHMKDSTFSAWTGLNDVNSEHTFLWTDGRGVHYTNWGKGYPGGRRSSLSYEDADCVVIIGGASNEAGKWMDDTCDGKRGYICQTRSDPSLTNPPATIQTDGFIKYGKSSYSLMRQKFQWHEAEIYCKLHNSLIASILDPYSNAFAWLQMETSNERVWIALNSNLTDSQYTWTDKWRVRYTNWAADEPKLKSACAYLDLDGYWKTAYCNESFYFLCKRSDEIPATEPPQLPGRCPESDHTAWIPFHGHCYYIESSYTRNWGQASLECLRMGSSLVSIESAAESSFLSYRVEPLKSKTNFWIGLFRNVEGMWLWINNSPVSFVNWNTGDPSGERNDCVALHASSGFWSNIHCSSYKGYICKRPKIIDAKPTHELLTTKADTRKMDPSKPSSSVAGVVIIVILLILTGAGLAAYFFYKKRRVHLPQEGAFENTLYFNSQSSPGTSDMKDLVGNIEQNEHAVI